(19) United States
(12) Patent Application Publication
(10) Pub. No.: US 2022/0213441 A1
(43) Pub. Date: Jul. 7, 2022

(54) EXTRACELLULAR MATRIX MATERIAL AND USES THEREOF

(71) Applicant: The Chinese University of Hong Kong, Shatin, New Territories (CN)

(72) Inventors: Anna Maria Blocki, Tai Po, New Territories (CN); Marisa Sofia De Oliveira Assunção, Tai Po, New Territories (CN)

(21) Appl. No.: 17/610,124

(22) PCT Filed: May 13, 2020
(86) PCT No.: PCT/CN2020/089991

§ 371 Date: Nov. 9, 2021

Related U.S. Application Data
(60) Provisional application No. 62/848,971, filed on May 16, 2019.

(51) Int. Cl. C12N 5/0775; A61K 35/28; C12N 5/071

(52) U.S. Cl. CPC C12N 5/0663; A61K 35/28; C12N 5/069; C12N 2533/80; C12N 2502/1358; C12N 2509/00; C12N 2509/10; C12N 2501/734; C12N 2533/70

(57) ABSTRACT

Provided are new methods for generating extracellular matrix material, compositions comprising the extracellular matrix material, and methods of using the extracellular matrix.

### RELATED APPLICATIONS

This application claims priority to U.S. Provisional Patent Application No. 62/848,971, filed May 16, 2019, the contents of which are hereby incorporated by reference in the entirety for all purposes.

### BACKGROUND OF THE INVENTION

It is known that engineered cell-derived extracellular matrices (ECMs) can be cell-rejuvenating¹, promote peripheral nerve growth in vitro² and even recapitulate the bone marrow niche sufficiently to promote hematopoietic progenitor cells' expansion³. The majority of previous studies on in vitro cell-derived ECM, however, focused on osteoblast- or chondrocyte-derived in vitro extracellular matrices and demonstrated that these extracellular matrices were not sufficient to induce terminal differentiation on their own. Nonetheless, they strongly augmented the differentiation of stem cells induced by standard differentiation factors^{4,5}. In vivo, several studies showed osteogenic potential of osteoblast-derived ECM^{6,7}, whereas in other studies such effects were not observed^{8,9}. Hence, although lineage specific in vitro ECM can be generated, the strength of its bioactivity is not stable, when generated by current standard culture methods. A major limitation of in vitro extracellular matrices is thus their instable bioactivity, caused by too little amounts of ECM that are deposited under standard culture and decreased even further after decellularization¹⁰.

Previously macromolecular crowding (MMC) has been used as a biophysical principle in in vitro biological systems, see, e.g., U.S. Pat. No. 9,809,798, WO2011108993A1, and WO2014077778 A1. The successful application of this biophysical principle was demonstrated by accelerated enzyme kinetics, such as procollagen C protease, leading to enhanced collagen I deposition¹¹ and collagenase activity¹² under MMC. It was also shown to increase supramolecular assemblies¹³, ECM cross-linking and stabilization¹¹, as well as ECM remodelling^{11,12.} Under MMC the amount of deposited ECM after a few days exceeds the amount of ECM, which can be accumulated within weeks under standard culture conditions, by several fold¹¹. More recent findings have shown that for some macromolecules, such as dextran sulfate (DxS), the effect on ECM deposition was not due to the accelerated molecular kinetics related to the increased fractional volume occupancy of DxS, but rather to the co-aggregation and co-precipitation of the macromolecules with ECM components¹⁴. For the ease of this invention all the methodologies enhancing ECM deposition by macromolecules are summarized as MMC.

It was shown that in vitro derived ECM generated under MMC was able to drive terminal differentiation of mesenchymal stem cells (MSCs) into adipocytes without the addition of any inductive factors. This is in contrast to other state-of-the-art studies utilizing cell-derived ECM generated without MMC^{4,5} and the study's own no-MMC ECM controls¹².

MMC effects in cell culture are not restricted to ECM formation. It has been shown that MMC could enhance proliferation in various cell types¹¹ and enabled the sourcing of hematopoietic pericytes from human peripheral blood^{14,15}. Nonetheless, the effect of MMC on the alteration of ECM-producing cells, such as their anti-inflammatory phenotype and the anti-inflammatory properties of their deposited ECM, were not investigated previously.

Pre-conditioning of MSCs activates their immunomodulatory and anti-inflammatory properties. These include pre-treatment with hypoxia or pro-inflammatory factors such as interferon-γ (IFNγ)¹⁶, lipopolysaccharide (LPS) or interleukin-1β (IL1β)¹⁷. Nonetheless, such pre-treatments have their own limitations, as accidental co-delivery of these pro-inflammatory factors might have adverse effects. In addition, over-exposure of MSCs to pro-inflammatory molecule LPS was shown to induce a pro-inflammatory phenotype¹⁷.

It has been shown previously that the addition of macromolecules can lead to changes in the ECM's properties, such as topography and mechanical properties¹⁴. Nonetheless, it hasn't yet been investigated if the addition of these macromolecules and their potential incorporation would lead to changes in ECM's bioactivity, such as its angiogenic potential.

The present disclosure relates to ECM-based biomaterials assembled by cells that have been activated to exhibit enhanced anti-inflammatory properties by MMC or a molecule known to exhibit anti-inflammatory properties, or the combination of both. Further, the present disclosure also relates to ECM-based materials assembled in the presence of macromolecules that exhibit enhanced pro-angiogenic properties. These culture conditions lead to the assembly of anti-inflammatory, immuno-modulatory and angiogenic ECMs in vitro. This process represents an entirely new method to manufacture cell-derived extracellular matrices with customized bioactivities, for example, anti-inflammatory and pro-angiogenic activities. Various applications of this new material are disclosed herein.

### BRIEF SUMMARY OF THE INVENTION

The present invention resides in the discovery that cells in culture can be stimulated to exhibit a desirable phenotype and that it is possible to customize the bioactivity of their assembled ECM by utilization of macromolecules. This renders the resultant ECM material particularly advantageous for applications such as wound healing and tissue repair or regeneration in a therapeutic context. Thus, in a first aspect, the invention provides a new method for generating an ECM-based material.

In some embodiments, a method is disclosed herein to obtain an ECM-based biomaterial comprising: a cell culture, for example, a culture of adhesive cells; supplementation of cell culture with glycosaminoglycans or carbohydrate-based hydrophilic macromolecules, or a combination thereof; maintaining the cell culture under conditions in which cells acquire an altered phenotype (e.g., altered expression of certain genes, especially anti-inflammatory factors) or assemble ECM with specific bioactivity (e.g. anti-inflammatory, pro-angiogenic); decellularization of the cell-derived ECM; and further processing of the cell-derived ECM into an applicable structure, which results in an ECM-based biomaterial with customized bioactivity.

In some embodiments of the invention, the cell culture contains ECM-producing cells. In some embodiments of the invention, the ECM-producing cells are mesenchymal stromal/stem cells. In some embodiments of the invention, the glycosaminoglycan is heparan sulfate, chondroitin sulfate, dermatan sulfate, keratan sulfate, hyaluronic acid, proteoglycans, derivatives therefrom, combinations thereof. In some embodiments of the invention, the glycosaminoglycan is hyaluronic acid. In some embodiments of the invention, the carbohydrate-based hydrophilic macromolecule is a polymer of glucose, sucrose or a combination thereof. In some embodiments of the invention, the carbohydrate-based hydrophilic macromolecule is the polymer Ficoll™70, Ficoll™400, polyvinyl pyrrolidone (PVP), dextran, dextran sulfate, polystyrene sulfonate, pullulan, or a combination thereof. In some embodiments of the invention, the cells are contacted with a mixture of carbohydrate-based hydrophilic macromolecules comprising Ficoll™70 and Ficoll™400.

In some embodiments of the invention, the supplementation of cell culture with glycosaminoglycans or carbohydrate-based hydrophilic macromolecules, or a combination thereof, induces an alteration of phenotype of the cells. In some embodiments of the invention, the alteration of phenotype is for example but not limited to the activation of an anti-inflammatory phenotype. In some embodiments of the invention, said anti-inflammatory phenotype can be identified by expression or secretion of anti-inflammatory factors such as, but not limited to, growth factors, cytokines, chemokines, exosomes or ECM components. In some embodiments of the invention, the anti-inflammatory factors are for example but not limited to transforming growth factor-β (TGF(3), hepatocyte growth factor (HGF), vascular endothelial growth factor (VEGF), fibroblast growth factor (FGF), insulin-like growth factor (IGF), epidermal growth factor (EGF), bone morphogenetic protein (BMP), granulocyte colony-stimulating factor (G-CSF), granulocyte-macrophage colony-stimulating factor (GM-CSF), stem cell factor-1 (SCF1), IL10 and IL6, monocyte chemoattractant protein-1 (MCP1), IL37, IL8, IL1 receptor α (IL1Rα), indoleamine 2,3-dioxygenase (IDO), prostaglandin E2 (PGE2) and tumor necrosis factor α-stimulated gene-6 (TSG6). In some embodiments of the invention, the anti-inflammatory factor is for example, but not limited to, IL10. In some embodiments of the invention, the supplementation of cell culture with carbohydrate-based hydrophilic macromolecules induces an alteration of the properties of the cell-assembled ECM. In some embodiments of the invention, the alteration of ECM's properties comprises, but is not limited to, changes in bioactivity such as enhanced pro-angiogenic properties. In some embodiments of the invention, the decellularization method lyses the cells, thereby producing a cell-free ECM. In some embodiments of the invention, the method for cell lysis includes osmotic shock, freeze-thawing cycles and/or bringing the cell culture in contact with lysing agents and combinations thereof. In some embodiments, the lysing agents are ionic, non-ionic and non-denaturating, zwitterionic detergents or chelating agents, nucleases and combinations thereof. In some embodiments of the invention, the lysing agents are deoxycholate (DOC), octylphenoxypolyethoxyethanol, 3-[(3-cholamidopropyl)dimethylammonio]-1-propanesulfonate (CHAPS), ethylenediaminetetraacetic acid (EDTA), DNAse or a combination thereof.

In a second aspect, the invention provides a new composition comprising the extracellular matrix material produced by the method described above and herein. In some embodiments, the cell-derived ECM-based biomaterial is further collected by, for example, but not limited to, uplifting, mechanical removal or solubilization. In some embodiments of the invention, the collected cell-derived ECM is incorporated or processed otherwise into an applicable structure, for example, a liquid, solid, emulsion, gel, paste, spray, nanoparticle, microcapsule, film, patch, bead, capsule, hydrogel, microbead, and moulded, printed, bio-printed structure, or a combination thereof. In some embodiments, the applicable structure is applied as a medicament for the treatment of a disease, which in some cases may be characterized by, for example, dysregulated tissue microenvironments. In some embodiments of the invention, the dysregulated tissue microenvironment is characterized by, for example, chronic inflammation and/or ischemia. In some embodiments of the invention, the cell-derived ECM or the applicable structure exhibits customized bioactivity. In some embodiments, the customized bioactivity is, for example, but not limited to, anti-inflammatory and/or pro-angiogenic properties. In some embodiments of the invention, the anti-inflammatory properties induce an anti-inflammatory phenotype in other cells. In some embodiments of the invention, the other cells are immune cells such as, but not limited to, monocytes, macrophages and T cells. In some embodiments of the invention, the immune cells are macrophages. In some embodiments of the invention, the induction of an anti-inflammatory phenotype in other cells is identified by down-regulation of pro-inflammatory markers or the up-regulation of anti-inflammatory markers, or a combination thereof. In some embodiments of the invention, the anti-inflammatory markers are, for example, but not limited to, IL10, pentraxin, PGE2, IL4 and IL13, VEGF, platelet-derived growth factor (PDGF), FGF, TGFβ, cluster of differentiation 206 (CD206) and pro-inflammatory markers are tumor necrosis factor-α (TNFα), IL12, IFNγ, IL6 and IL1β. In a preferred embodiment of the invention the anti-inflammatory marker is IL10.

In some embodiments of the invention, the pro-angiogenic properties induce a pro-angiogenic behavior in other cells. In some embodiments of the invention the other cells include, but are not limited to cardiovascular, immune, neural, musculoskeletal, renal, skin and adrenal cells. In some embodiments of the invention, the other cells are vascular and perivascular cells, but not limited to, endothelial cells, fibroblasts, and pericytes. In some embodiments of the invention, the angiogenic cells are endothelial cells. In some embodiments of the invention the angiogenic behavior is determined by enhanced and or accelerated vessel sprouting (angiogenesis), vasculogenesis, arteriogenesis, vessel maturation and stabilization. In a preferred embodiment of the invention, the angiogenic behavior is increased vessel sprouting.

In yet another aspect, the present invention provides a method for using the extracellular matrix material produced by the method described above and herein. In some embodiments, the method is used for enhancing wound healing or tissue repair/regeneration, including the step of placing the extracellular matrix material of this invention or the composition comprising the extracellular matrix material at a site of tissue damage, e.g., within a patient's body. In some embodiments, the tissue damage is caused by an injury, such as one inflicted by external force, or a disease or an internal condition of the patient. In some embodiments, the disease is biliary ischemia, bone-related ischemia, cerebral ischemia, colonic ischemia, coronary ischemia, foot-related ischemia, hepatic ischemia, mesenteric ischemia, myocardial ischemia, optical nerve ischemia, retinal ischemia and spinal ischemia, peripheral artery disease, myocardial infarction, chronic wounds, or osteoarthritis. In some embodiments, the disease is myocardial infarction. In some embodiments, the disease is chronic wounds. In some embodiments, the disease is osteoarthritis.

In a related aspect, the present invention provides a use of the extracellular matrix material produced by the method described above and herein, or a composition comprising the extracellular matrix material. In some embodiments, the extracellular matrix material produced by the method described above and herein or a composition comprising the extracellular matrix material is used for manufacturing a therapeutic material for the purpose of promoting wound healing or tissue repair/regeneration, which may be placed at a site of tissue damage, e.g., within a patient's body. In some embodiments, the tissue damage is caused by an injury, such as one inflicted by external force, or a disease or an internal condition of the patient. In some embodiments, the disease is biliary ischemia, bone-related ischemia, cerebral ischemia, colonic ischemia, coronary ischemia, foot-related ischemia, hepatic ischemia, mesenteric ischemia, myocardial ischemia, optical nerve ischemia, retinal ischemia and spinal ischemia, peripheral artery disease, myocardial infarction, chronic wounds, or osteoarthritis. In some embodiments, the disease is myocardial infarction. In some embodiments, the disease is chronic wounds. In some embodiments, the disease is osteoarthritis.

### BRIEF DESCRIPTION OF THE DRAWINGS

FIG. 1 Deposition of ECM components by MSCs in the presence of exogenously added high molecular weight hyaluronic acid (HMWHA) and MMC. MSCs were cultured for 2 to 6 days with HMWHA (1.5-1.75 MDa) with a concentration ranging from 0 to 1000 μg/ml in the presence or absence of a MMC (Ficoll 70 kDa at 37.5 mg/ml and Ficoll 400 kDa at 25 mg/ml). Immunostaining for ECM components and microscopy at low magnification revealed amplified deposition of A) hyaluronic acid, B) fibronectin, and C) collagen I. n=3 independent runs.

FIG. 2 Quantification of the area covered by ECM components deposited by MSCs in the presence of exogenously added HMWHA and MMC. MSCs were cultured for 2 to 6 days with HMWHA (1.5-1.75 MDa) with a concentration ranging from 0 to 1000 μg/ml in the presence or absence of MMC (Ficoll 70 kDa 37.5 mg/ml and Ficoll 400 kDa 25 mg/ml). The fluorescence pictures taken from immuno-stained ECM components account for 14% of the total area of the cell culture area. As such, they were used to representatively quantify the area covered by A) hyaluronic acid, B) fibronectin and C) collagen I, using Image J software. n=3 independent runs. * p<0.05, #p<0.05, ** p<0.01.

FIG. 3 MMC enhanced fibronectin (FN) and collagen I deposition into the cell layer. (A) Day 6 cell layer samples of human bone marrow MSC's cultures, optionally supplemented with 5-500 μg/ml HMWHA (1.5-1.75 MDa) and MMC (Ficoll 70 kDa at 37.5 mg/ml and Ficoll 400 kDa at 25 mg/ml), were collected into sodium dodecyl sulphate-polyacrylamide gel electrophoresis (SDS-PAGE) sample buffer. The total protein extracts from the cell layer were analysed by western blot for fibronectin and GAPDH. Independent experimental runs n=3 (B) Day 6 cell layers and supernatants (culture media) samples were digested by pepsin and the remaining collagenous proteins were visualized by silver staining after being separated on an SDS-PAGE gel. Independent experimental runs n=3.

FIG. 4 Human bone marrow MSCs were cultured for 2 days in the presence of HMWHA (500 μg/ml) and MMC (Ficoll 70 kDa at 37.5 mg/ml and Ficoll 400 kDa at 25 mg/ml). MSC-derived messenger RNA (mRNA) was collected from the cell layers and analysed by reverse transcriptase-quantitative polymerase chain reaction (RT-qPCR). The obtained cycle quantification (Ct) values for IL10 were normalized to GAPDH Ct values and expressed as fold-change to MSCs cultured without HMWHA and MMC (Control MSCs). *p<0.05. MSCs expressed increased levels of IL10, when cultured in the presence of HMWHA and MMC.

FIG. 5 Phase contrast images of human bone marrow MSC-derived ECM assembled in the presence of HMWHA (500 μg/ml) and MMC (Ficoll 70 kDa at 37.5 mg/ml and Ficoll 400 kDa at 25 mg/ml) for 6 days before (left) and after decellularization by deoxycholate and DNase (right).

FIG. 6 Macrophages were seeded on tissue culture polystyrene (TCP), 1% (wt/v) gelatin, control MSC-derived ECM and on ECM assembled in the presence of HMWHA (500 μg/ml) or MMC (Ficoll 70 kDa at 37.5 mg/ml and Ficoll 400 kDa at 25 mg/ml). The cells were cultured for 24 h and then pulsed with 10 ng/ml LPS and 5 ng/ml of IFNγ for 30 minutes. Non-pulsed macrophages on TCP were used as non-polarized control. Conditioned medium was analysed by enzyme-linked immunosorbent assay (ELISA) for human TNFα after 24 h. #p<0.05, ** p<0.01, *** p<0.001, **** p<0.0001, ####p<0.0001. Cell-derived ECMs assembled in the presence of HMWHA or MMC alone and HMWHA in combination with MMC completely inhibited pro-inflammatory M1 polarization of macrophages.

FIG. 7 Endothelial cells spheroids were seeded on tissue culture polystyrene (TCP), on unmodified MSC-derived ECM (cECM) or on MSC-derived ECM assembled in the presence of dextran sulfate (500 KDa, 10 μg/ml) (DxS-ECM), while embedded in collagen I hydrogel. The cumulated endothelial sprout length (de novo formed vascular-like sprouts) was quantified after 24 h in contact with the ECM-based biomaterials or TCP. *** p<0.001, **** p<0.0001. DxS-ECM-based biomaterials significantly increased endothelial cell sprouting.

### DEFINITIONS

The term “activating” or “activation,” as used herein, refers to any detectable positive or enhancing effect on a target biological or pathological process, such as the expression of one or more pre-determined genes, proliferation of cells, exhibition of a particular morphology, and the like. Typically, an activation is reflected in an increase of at least 10%, 20%, 50%, 100%, or 2 times, 3 times, 5 times, or up to 10 times, or even higher in a feature characteristic of the target process (e.g., the rate of cell proliferation or gene expression) when compared to a control. Similarly, the term “inhibiting” or “inhibition,” as used herein, refers to any detectable negative or suppressing effect on a target biological or pathological process. Typically, an inhibition is reflected in a decrease of at least 10%, 20%, 30%, 40%, or 50% in a feature characteristic of the target process (e.g., the rate of cell proliferation or gene expression) when compared to a control.

As used herein, “a stimulant altering the cells' phenotype” refers to a substance that can, upon contact with target cells, affect the cells' characteristics such as causing activation or inhibition of the level of gene expression, protein secretion, cell proliferation, adhesion, migration, contact inhibition, and detectable changes in morphology, etc.

The term “effective amount,” as used herein, refers to an amount of a substance that produces detectable biological effects for which the substance is applied. The effects may include, but are not limited to, characteristics of cells such as increase or decrease in the level of gene expression, protein secretion, cell proliferation, adhesion, migration, contact inhibition, as well as detectable changes in morphology, etc.

A “glycosaminoglycan” is a long unbranched polysaccharides consisting of a repeating disaccharide unit. Except for keratan, the repeating unit consists of an amino sugar (N-acetylglucosamine or N-acetylgalactosamine) along with a uronic sugar (glucuronic acid or iduronic acid) or galactose.

The term “carbohydrate-based hydrophilic macromolecule” is used herein in reference to any macromolecule that comprises at least a substantial carbohydrate portion and generally exhibits a hydrophilic profile.

As used herein, the term “administration” encompasses any means of delivering or applying a substance, e.g., an agent with desired therapeutic or prophylactic effects, to a subject in need of the benefit of such therapeutic or prophylactic effects, which may include but is not limited to, systemic, regional, and local applications. Examples of “administration” include injection (such as by subcutaneous, intramuscular, intravenous, or intraperitoneal means), oral ingestion, intake through the nasal cavity or through the eyes or ears, inhalation, transdermal delivery, topical application, and direct deposit via any one of body cavities or surgical incisions, etc.

The terms “pharmaceutically acceptable excipient” and “physiologically acceptable excipient” may be used interchangeably to refer to an inert substance that is included in the formulation of a composition containing an active ingredient or a main structural component to achieve certain characteristics, such as more desirable pH, solubility, stability, bioavailability, texture, consistency, appearance, flavor/taste, viscosity, etc., but in itself does not negatively impact the intended therapeutic or prophylactic effects of the active ingredient or main structural component.

The term “tissue,” as used herein, refers to an ensemble of cells that are similar in their biological attributes, such as morphology and biological activity, and are from the same origin, such that these cells together carry out a specific function. An “organ” is a collection of different tissues joined in a structural unit to serve a common function.

The term “about,” as used herein, describes a range of plus or minus 10% from a recited value. For example, a value of “about 10” can be any value within the range of 10±1, i.e., between 9 to 11.

### DETAILED DESCRIPTION OF THE INVENTION

### I. Introduction

The present invention provides a novel material for tissue healing and a method for manufacturing this material, which is characterized as a biomaterial based on ECM. This ECM can be cell-derived, and this new ECM-based biomaterial can promote tissue healing by exhibiting anti-inflammatory and/or pro-angiogenic properties and guiding dysregulated tissue microenvironments towards healing and regeneration.

More specifically, this disclosure relates to (1) a biomaterial based on ECM exhibiting customized bioactivity, inferring desired properties such as, but not limited to, anti-inflammatory, immuno-modulatory and pro-angiogenic bioactivity; and (2) a process for manufacturing the ECM-based materials. Advantageously, in some embodiments, the biomaterial-based ECM can alter cellular responses, induce polarization of macrophages towards a pro-healing M2 phenotype, inhibit polarization of macrophages towards a pro-inflammatory M1 phenotype, and induce endothelial cell sprouting. In other embodiments, the process for manufacturing the biomaterial can alter the phenotype of ECM-producing cells to induce an anti-inflammatory phenotype.

In addition to the forgoing attributes, the ECM-based biomaterial possesses numerous benefits over conventional and experimental approaches to treat diseased dysregulated tissue environments. The benefits include that the bioactive material can be stored and thus applied off-the-shelf, while exhibiting sufficient complexity in its bioactivity to affect intricate biological processes and thereby promote tissue healing and regeneration. Furthermore, another benefit in some embodiments is that the ECM-based biomaterial can be of human origin, while manufactured in sufficient amounts with a stable and reproducible bioactivity, which can be customized to a specific clinical application.

### II. Production of Extracellular Matrix Material

The present invention provides a novel method for producing an extracellular matrix material that has desirable biological activities, such as anti-inflammatory and pro-angiogenic activities. The method includes these steps: first, culturing cells in the presence of an effective amount of a stimulant altering the cells' phenotype and under conditions permissible for the cells to produce an ECM, either by forming cellular aggregates or by adhering to the surface of a solid substrate or semi-solid substrate, or to produce an ECM within the framework of a solid (e.g., mesh-like) substance or a semi-solid substance to form an ECM substantially contained within the framework; and second, obtaining extracellular matrix material formed by the cells by isolating the extracellular matrix material from the cell culture,

A variety of cell types can be used in the production of the extracellular matrix material of this invention. In some cases, it is preferable that an adhesive cell type (which adheres to a solid or semi-solid substrate) be used in the process. For example, suitable cells may be stem or stromal cells such as mesenchymal stem/stromal cells or a mixture thereof. In some cases, the ECM-producing stromal cells are liver-derived cells, pancreas-derived cells, umbilical cord-derived cells, umbilical cord blood-derived cells, brain-derived cells, spleen-derived cells, bone marrow-derived cells, adipose-derived cells, cells derived from induced pluripotent stem cell (iPSC) technology, cells derived from embryonic stem cells, genetically engineered cells, pluripotent cells, multipotent cells, neural cells, astrocytes, hepatocytes, fibroblasts, mesenchymal cells, epithelial cells, endodermal cells, pericytes, cardiomyocytes, cardiomyocyte progenitor cells, hematopoietic cells, endothelial cells, endothelial progenitors, smooth muscle cells, keratinocytes, stem cells and progenitors cells, or mixtures thereof.

In order to achieve the particularly desired biological activities, such as anti-inflammatory and/or pro-angiogenic activities, in the extracellular matrix material of this invention, one or more stimulants may be introduced into the cell culture in an effective amount for achieving such desired biological activities. For instance, the cell culture used for generating an extracellular matrix material of this invention is supplemented with a glycosaminoglycan, a carbohydrate-based hydrophilic macromolecule, or a combination thereof. In some cases, the glycosaminoglycan is heparan sulfate, chondroitin sulfate, dermatan sulfate, keratan sulfate, hyaluronic acid, proteoglycans carrying these glycosaminoglycans, derivates therefrom, or any one of the possible combinations thereof.

For example, the glycosaminoglycan is hyaluronic acid, which may be human or animal tissue-derived or derived from bacterial or other cell culture. In some cases, the hyaluronic acid has a molecular weight range of about 2 kDa to about 10,000 kDa, high molecular weight of about 1,500 kDa to about 2,000 kDa or 1,600 kDa. In some cases, the glycosaminoglycan is added into the cell culture at a concentration range from about 0.5 μg/ml to about 5000 μg/ml, about 5 μg/ml to about 1000 μg/ml, or at a concentration of about 500 μg/ml. In some cases, the carbohydrate-based hydrophilic macromolecule used in the method is a polymer of glucose, sucrose, or a combination thereof. For example, the polymer is Ficoll™70, Ficoll™400, polyvinyl pyrrolidone (PVP), dextran, dextran sulfate, polystyrene sulfonate, pullulan, or a combination thereof. In some cases, the cell culture is contacted with a mixture of carbohydrate-based hydrophilic macromolecules comprising Ficoll™70 and Ficoll™400: for example, the Ficoll™70 is at a concentration range of from about 7.5 mg/ml to about 100 mg/ml, and the Ficoll™400 is at a concentration range of from about 2.5 mg/ml to about 100 mg/ml; or the Ficoll™70 is at a concentration of about 37.5 mg/ml and the Ficoll™400 is at a concentration of about 25 mg/ml. In some cases, the cell culture is contacted with a mixture of carbohydrate-based hydrophilic macromolecules comprising Dextran sulfate: for example, dextran sulfate with a molecular weight of 500 kDa is at a concentration range of from about 0.10 μg/ml to about 10 mg/ml; or the dextran sulfate (500 kDa) is at a concentration of about 10 μg/ml.

Following stimulation of the cultured cells by adding into the culture an effective amount of one or more a substances capable of altering the cells' phenotype (e.g., increasing or decreasing the expression of at least one pre-determined gene, increasing or decreasing secretion of at least one pre-determined protein), after an adequate length of time (e.g., at least 12 hours, 24 hours, 36 hours, or 48 hours or up to 3, 4, 5, 6, 7, 8, 9, or 10 days) the altered phenotype can be confirmed (e.g., using immunoassays detecting the expression or secretion level of a target protein) and ECM molecules assembled in the in vitro cell culture can be detected (for example, by detecting ECM molecules such as glycosaminoglycans, hyalectans, proteoglycans, collagens, elastin and elastin-associated molecules, laminins, matricellular proteins, especially fibronectin, hyaluronic acid and collagen I. In some cases, the cells are activated to exhibit an anti-inflammatory phenotype, which, for instance, may be detected by increased mRNA and/or protein levels of anti-inflammatory factors such as growth factors, cytokines, chemokines, exosomes or ECM components, including but not limited to TGFβ, HGF, VEGF, FGF, IGF, EGF, BMP, G-CSF, GM-CSF, SCF1, IL10 and IL6, MCP1, IL37, IL8, IL1Ra, IDO, PGE2 and TSG6. IL10 is a preferred example. Due to the stimulation of the cultured cells, the extracellular matrix material of this invention has anti-inflammatory properties. For example, the anti-inflammatory properties can induce an anti-inflammatory phenotype in other cells, including immune cells such as monocytes, macrophages, and T cells, especially macrophages. The anti-inflammatory phenotype can be identified by down-regulation of a pro-inflammatory marker or the up-regulation of anti-inflammatory marker, or combinations thereof. For example, the anti-inflammatory markers are IL10, pentraxin, PGE2, IL4 and IL13, VEGF, PDGF, FGF, TGFβ and CD206 and pro-inflammatory markers are TNFα, IL12, IFNγ, IL6, and IL1β. TNFα is a preferred example for a pro-inflammatory marker.

In some cases, the supplementation of cultured cells with macromolecules produces an extracellular matrix-based biomaterial with enhanced pro-angiogenic properties. The pro-angiogenic properties can be verified, for example, by enhanced new vessel formation by processes such as endothelial sprouting, vasculogenesis and/or arteriogenesis. Enhanced vasculogenesis includes, for example, longer vessel stability, formation of denser vascular networks, formation of thicker vessels, formation of more vessels.

With the desired biological properties, the extracellular matrix material produced by the cells can then be harvested, for example, by peeling or uplifting the material from the solid substrate using mechanical force or by removing the solid or semi-solid substrate when the cells have formed the ECM within the substrate framework or by solubilization before incorporation or processing further into an applicable structure. Exemplary structure includes a liquid, solid, emulsion, gel, microparticle, nanoparticle, microcapsule, film, patch, bead, capsule, hydrogel, microbead, and molded, printed, bio-printed structure, or a combination thereof.

Optionally, a decellularization step can be taken to remove all or nearly all (e.g., at least 80%, 90%, 95%, 98%, 99% or more) cells present within the extracellular matrix material to produce a cell-free or essentially cell-free (e.g., at least 80%, 90%, 95%, 98%, 99% or higher) extracellular matrix material. Various methods can be used to lyse the cells, including the use of osmotic shock, one or more freeze-thaw cycles, one or more lysing agents, and any combinations thereof. For instance, the lysing agent may be an ionic, non-ionic and non-denaturating, zwitterionic detergent or chelating agent, nuclease, or a combination thereof: e.g., the lysing agent may be deoxycholate, octylphenoxypolyethoxyethanol, 3-[(3-cholamidopropyl)dimethylammonio]-1-propanesulfonate (CHAPS), Ethylenediaminetetraacetic acid (EDTA), DNAse, or a combination thereof.

Upon further processing of the extracellular matrix material, it can be used in a variety of therapeutic applications for treating conditions involving tissue damage or injury, which may be caused by mechanical force (external injury) or disease (internal cause), or a combination thereof resulting in a dysregulated tissue microenvironment. The extracellular matrix material of this invention or a composition comprising the extracellular matrix material is typically applied directly to the site of tissue damage so as to promote and enhance healing and/or regeneration of injured tissue. In some cases, the use of the extracellular matrix material of this invention may be used for treating a disease such as biliary ischemia, bone-related ischemia, cerebral ischemia, colonic ischemia, coronary ischemia, foot-related ischemia, hepatic ischemia, mesenteric ischemia, myocardial ischemia, optical nerve ischemia, retinal ischemia and spinal ischemia, peripheral artery disease, myocardial infarction, chronic wounds, osteoarthritis, with the treatment of myocardial infarction, osteoarthritis, or chronic wounds being most promising.

### III. Therapeutic Applications of Extracellular Matrix Material

The invention also provides methods for using the extracellular matrix material produced by the methods described above and herein for various applications in the therapeutic contexts.

### A. Myocardial Infarction

Limitations of established treatments for myocardial infarction

Acute myocardial infarction primary occurs due to occlusion of a coronary artery. Current treatment options and interventions mainly focus on the re-establishment of blood flow within the affected area using drugs (anti-platelet drugs such as aspirin), as well as catheter-based (angioplasty, stenting) and surgical intervention (bypass). Other measures to protect the ischemic myocardium immediately after occurrence of the infarct and also during chronic heart failure include the administration β-adenoreceptor blockers and angiotensin-converting-enzyme inhibitor. These drugs decrease the oxygen demand of the cardiac tissue⁴⁵.

None of the current treatment options focus on the reparation or regeneration of the affected tissue. However, the myocardium is an aerobic high-performance tissue that experiences an irreversible damage (necrosis=tissue death) within hours after onset of ischemia⁴⁶. The necrotic tissue will cause a strong inflammatory and persistent response as well as a decreased oxygen supply, also affecting the tissue surrounding the necrotic area (penumbra). The opening of the coronary artery improves the salvage of the injured tissue, however it also leads to a burst of oxidative stress causing further tissue necrosis^{3,47}.

This in combination with an increased mechanical stress will lead to the expansion of the infarcted area. This area will be replaced by a scar over the course of time, which cannot partake in the cardiac pumping function. As a result, an increasing scarring area will ultimately lead to chronic heart failure^{3,47}.

In conclusion, although current established treatments reduce mortality, they fail to prevent the expansion of the infarcted area and thus rescue of the tissue at risk by improving the chronically inflamed, ischemic and dysregulated microenvironment.

### Limitations of Experimental Approaches for the Treatment of Myocardial Infarction

Several experimental approaches exist that attempt to address the limitations of established treatment options. Strategies to replace lost cardiomyocytes include (reviewed in²²):
Activation of Endogenous Cardiomyocyte Proliferation
   Estimated cardiomyocyte cell renewal<1% annually, decreasing with age.
   First indications of possible reactivation and enhancement of cardiomyocyte proliferation in small animal models.
   Induced by genetic modification, mainly targeting the cell cycle 4 risk of teratogenicity.
Activation/Stimulation of Cardiac Progenitor Cells
   Current strategies did not lead to new cardiomyocytes in significant numbers.
Exogenous Cardiomyocyte Replacement
   iPSC-derived cardiomyocytes are transplanted in large numbers (estimated to 10⁹-10¹⁰ cells) into the infarcted myocardium, proof-of-concept in large animal studies, however lethal arrhythmias observed in all animals.
   Although patients' own cells can be generated, the production scale of several hundred million surviving transplanted cells remains a challenge and is very cost-intensive.
In Vivo Reprogramming of Fibroblasts into De Novo Cardiomyocytes
   Direct in vivo genetic reprogramming of fibroblasts into cardiomyocytes.
   First promising data in mouse model.
   Remaining challenges include: Selectivity of targeting the heart only, achieving maturation in terms of structure and function in reprogrammed cells, functional integration of reprogrammed cells into existing tissue.

Although current therapies to replace lost cardiomyocytes are very promising, they are still facing many short-comings. One of the major ones, which is shared by all approaches, is the hostile microenvironment new cardiomyocytes are exposed to. This strongly inflammatory environment causes the expansion of the infarct and continuously induces death of the cardiomyocytes surrounding the primary infarct area. This will, of course, also negatively affect transplanted or reprogrammed cardiomyocytes. When exposed to the same hostile microenvironment all cardiomyocytes, the pre-existing and the new ones, will suffer the same fate. This is also the reason why such high cell numbers are required to achieve any effect in exogenous cardiomyocyte replacement.

Therefore, it is a prerequisite to modulate the microenvironment, not only to impair the expansion of the infarct and to rescue the tissue at risk, but also to prepare a cardiomyocyte-supportive microenvironment for new cardiomyocytes.

Strategies targeting the modulation of the microenvironment in the infarct area include:
Enhancing Angiogenesis²³
   Delivery of single factors (e.g. VEGFA) or gene therapy: clinical trials unsuccessful.
Immunomodulation²³
   Immunosuppressive agents: clinical trials unsuccessful.
Adult Cell-based Therapy²⁴
   Limited engraftment and cell survival in infarcted area²⁵.
   No significant long-term improvement in clinical trials²⁵.
Biomaterials for Cardiac Repair
   Injectable hydrogels and heart patches²⁶: Biomaterials for cardiac repair are mainly investigated in pre-clinical studies, where they have shown improvement in functional and cardiac remodelling post myocardial infarction. They provide mechanical support to the moving tissue and can co-deliver bioactive molecules and cells to promote healing.
      Although cellular engraftment can be facilitated, cells still encounter a hostile environment, thus limiting their survival.
      Selected bioactive components, which are delivered in such biomaterials are also insufficient to divert the complex biological processes, such as chronic inflammation, towards healing.
      Such biomaterials are often fabricated from synthetic or natural non-mammalian (e.g. alginate) components and as such can be recognized as foreign materials by the patient's own immune system, thus causing additional adverse reactions²⁷.
   Tissue-derived ECM can be manufactured into injectable hydrogels and patches and is capable to address many of the limitations faced by other biomaterials (see above). It is derived from mammalian sources (e.g. human or porcine) and has an intrinsic complexity in its structure and bioactivity. Indeed, tissue-derived ECM²⁸⁻³⁰ was demonstrated to improve cardiac healing in various pre-clinical experimental approaches²⁸⁻³⁰.

### B. Diabetic Chronic Wounds

Clinically established therapies comprise of off-loading, repeated debridement, antibiotic treatments and various dressings. In addition, reperfusion strategies (e.g., angioplasty) help to restore major blood flow³². Other FDA-approved approaches based on bioengineered skin substitutes (Dermagraft® and Apligraft®) experience a short half-life, as the dysregulated environment also negatively affects the implanted cells³². Hence, current treatment approaches are insufficient to treat chronic wounds, as none of them sufficiently targets the hostile chronically inflamed, ischemic and dysregulated environment.

Experimental therapeutic approaches: Various strategies have been explored to improve the harsh microenvironment in diabetic chronic wounds and thereby augment wound healing, including growth factor treatment, application of various bioengineered scaffolds, cell-based therapies and combinations thereof.

Growth factors have a very short half-life and thus do not remain in the wound bed long enough to exhibit a significant effect. Their retention can be prolonged by being delivered in a scaffold (e.g., Regranex®). Nevertheless, supra-physiological doses can lead to dramatic side effects, such as cancer. Further, single growth factors do not exhibit the required complexity in bioactivity to correct the multiple molecular processes in chronic wounds⁵.

Cell-based therapy offers a more holistic approach, where cells sense and respond to the microenvironment by secreting a wide range of paracrine factors locally. Mainly adult MSCs from bone marrow and adipose tissue have been investigated⁵. MSCs are anti-inflammatory and pro-angiogenic⁵⁴ and promote a shift in the wound microenvironment from the inflammatory to the proliferation phase⁵. Nevertheless, cell-based therapies still face various limitations such as limited engraftment and survival upon implantation⁵⁵.

Tissue engineered scaffolds, consisting of natural components, synthetic components or a combination of both (semi-synthetic), were often utilized to mimic certain pro-regenerative features of the native ECM. Nevertheless, these scaffolds fail to recapitulate the complex structure, which is necessary to amend the hostile wound microenvironment⁵⁶.

The ECM consists of a complex bioactive assembly of fibrillar proteins with associated components such as cytokines. The accurate organization of these components allows the ECM to harness their full complex bioactive strength and ensures long-term activity³⁶. Human decellularized skin matrices were shown to significant accelerate healing and closure of diabetic wounds in clinical trials³¹. The limited availability of human cadaveric tissue often also lead the use of animal tissue-derived ECM as an alternative source, which also had beneficial effects³². Nevertheless, tissue-derived ECM faces many limitations such as risk of disease transmission, limited availability of human tissue, immunological rejection of animal-derived products and the inability to customize the ECM's bioactivity³⁷.

### C. Osteoarthritis³⁸

### Established Therapeutic Approaches

Osteoarthritis treatments involve physical measures, drug therapy and surgery. Surgery is only considered for severe cases when conservative therapy is ineffective because of the invasive trauma and higher risks. Arthroscopic irrigation and debridement provide a certain degree of pain relief but are not beneficial for long-term recovery. Drilling and microfracture techniques aim at penetrating the subchondral plate to induce bone marrow stromal cells for spontaneous repair, but the repaired tissue has inferior mechanical properties and consists of fibrocartilage. Total joint replacement/arthroplasty is regarded as the best orthopedic surgery for advanced osteoarthritis. It can potentially reduce pain and improve joint function. Unfortunately, arthroplasty is not recommended for young patients, as the artificial implant has a finite lifespan (usually 10-15 years). In addition, the long-term results of arthroplasty differ significantly.

Pharmaceutical therapy is the most commonly used osteoarthritis treatment option aimed mainly at pain relief and anti-inflammation. The traditional osteoarthritis drugs are limited to control osteoarthritis symptoms, but none can reverse the damage in the osteoarthritis joint. Additionally, traditional drugs are always overwhelmed by their high incidence of adverse effects.

### Biologics

The unsatisfactory effects and unacceptable side effects associated with traditional osteoarthritis drugs warrant a continued search for potential new medications. Although few of them have received the regulatory approval for routine clinical use, a variety of new osteoarthritis drugs have shown promising results in clinical trials. On the basis of the potential therapeutic targets, they can be classified as chondrogenesis inducers, osteogenesis inhibitors, matrix degradation inhibitors, apoptosis inhibitors, and anti-inflammatory cytokines. Some biologics such as BMP7 showed encouraging first results, whereas others, such as IL1β inhibitor showed no improvement or even adverse effects such as in the case of β-nerve growth factor. Again, as deducted from other applications, such as in myocardial infarction or chronic wounds, single biological factors lack the necessary complex bioactivity to sustainably affect complex biological processes such as chronic inflammation.

### Cell-based Therapy

First described by Brittberg et al.⁵⁷ autologous chondrocyte implantation/transplantation (ACI/ACT) is widely used in clinical practice and more than 15,000 patients have received this treatment worldwide. Clinical outcomes enhanced osteochondral defect repair and formation of de novo hyaline cartilage. Reported adverse effects in about 50% of the patients were periosteal hypertrophy and intra-articular adhesions. Hence, this cell-based treatment is considered a reasonable treatment of cartilage defects.

However, cartilage damage with generalized osteoarthritis was an exclusion criterion for treatment. This is because ACI is applicable to localized cartilage defects surrounded by healthy cartilage. Osteoarthritis cartilage, however, often affects the adjacent areas and disturbs the homeostasis of the whole joint cavity. In this degenerative microenvironment, the implanted chondrocytes will undergo undesired dedifferentiation or apoptosis, therefore undermining efficacy.

Other cells, such as MSCs were investigated as well. Although a reduction of pain score was recorded, inconclusive data in the long-term outcome and dedifferentiation of MSCs remain to be addressed.

### Tissue Engineering Approaches

Cell-carrying scaffolds are being investigated for their ability to enhance the engraftment of cells in the lesion side. The results are often better than cells alone, although adverse effects were reported. In general, the degenerated environment still impairs cell survival and promotes cellular dedifferentiation.

Cell-free scaffolds delivering bioactive molecules are also being investigated, although they face the same limitations as growth factor therapy (see above) and were reported to be inferior to cell-based therapy.

Current therapies for diseases with a chronically inflamed dysregulated microenvironment focus on the treatment of symptoms, revascularization (in case of ischemic diseases) and secondary effects such as infection management (in case of chronic wounds). None of the established therapies successfully addresses the hostile microenvironment. Experimental approaches in pre-clinical or clinical studies attempt either to address the diseased microenvironment or to induce regeneration, but were not successful so far.

A broad downregulation of inflammation also impairs healing, as a specific inflammatory response is necessary for healing. Biologics delivered as growth factors, either in the form of proteins (by itself or in tissue engineered scaffolds) or as gene therapy, do not have sufficient bioactive complexity to amend the dysregulated microenvironment and turn it into a pro-healing one. Since such factors are delivered in supra-physiological doses, they also introduce many risks and adverse effects.

Biomaterials based on tissue-derived ECM intrinsically exhibit sufficient complex bioactivity and pre-clinical experiments have shown promising results. Nonetheless, tissue-derived ECM faces many limitations in clinical application such as risk of disease transmission, limited availability of human tissue, immunological rejection of animal-derived products and the lack of bioactivity customization. Its complexity and fixed composition confounds our understanding of the mechanism of action, thus diminishing the predictability of the ECM's therapeutic effect.

Cell-based therapy offers a more holistic approach, where cells sense and respond to the microenvironment by secreting a wide range of paracrine factors locally. MSCs appear to be promising due to their anti-inflammatory and immuno-modulatory properties. These can shift a dysregulated wound microenvironment into a pro-healing one. Unfortunately, cell-based therapies still face various limitations such as limited engraftment, low survival upon implantation, dedifferentiation and have provided very limited success so far.

By utilizing this invention, the extracellular matrix material, one is able to address the limitations of these experimental approaches. The ECM consists of a complex assembly of fibrillar proteins with associated bioactive components. The accurate organization of these components is a prerequisite to harness their full bioactive strength and ensure long-term stability. As the cell-derived ECM partially recapitulates the complex biological machinery of the native tissue environment¹⁰, it is envisioned that MSC-derived ECM will exceed its soluble counterpart in terms of bioactivity and long-term stability. Hence, by customizing MSC-derived ECM in vitro, one is potentiating the whole repertoire of the MSCs' environment-modulating properties.

In particular, the extracellular matrix material intrinsically exhibits the necessary bioactive complexity to amend and guide complex biological processes. By (1) utilizing the appropriate cell type (MSCs), which was already shown to exhibit the necessary bioactivity (anti-inflammatory and pro-angiogenic); (2) inducing sufficient ECM deposition with a stable bioactivity by using MMC; and (3) potentiating the bioactivity of the deposited ECM by choosing the right factors (HMWHA and/or MMC) during ECM assembly, it is possible to deposit a strongly pro-angiogenic properties and/or activate a strongly anti-inflammatory phenotype in MSCs, which translates into a strongly anti-inflammatory deposited ECM. This extracellular matrix material has the ability to completely block M1 polarization of macrophages and thus the potential to amend the hostile chronically inflamed microenvironment.

### EXAMPLES

The following examples are provided by way of illustration only and not by way of limitation. Those of skill in the art will readily recognize a variety of non-critical parameters that could be changed or modified to yield essentially the same or similar results.

### Example 1

Human bone marrow MSCs (Millipore; Lonza) were seeded between passage 6 and 9 at 6,500 cells per cm² in TCP plates at 0.3 ml volume per cm². The cells were allowed to attach for 24 h in Dulbecco's modified eagle medium (DMEM) with 10% fetal bovine serum (FBS) and 1% Penicillin/Streptomycin (P/S), after which the medium was exchanged for induction medium used to promote ECM assembly.

Induction of MSCs was done in DMEM supplemented with 0.5% FBS and 0.1 mM ascobic acid, 37.5 mg/ml Ficoll 70 kDa and 25 mg/ml Ficoll 400 kDa, as well as HMWHA (1.5-1.75 MDa, 500 μg/ml). Alternatively, MSCs were cultured in DMEM supplemented with 0.5% FBS and 0.1 mM ascobic acid and dextran sulfate (500 kDa 10 μg/ml). Cells were cultured for a maximum of 6 days without medium change and were then decellularized. For this, cells were carefully washed with phosphate buffered saline (PBS) twice at room temperature. Plates were placed on ice and washed with 0.5% sodium deoxycholate (DOC in water; Sigma) containing 0.5× protease inhibitor (from 400× stock in dimethyl sulfoxide) for 15 minutes. This solution was then replaced by 0.5% DOC in water for 10 minutes at room temperature. The solution was then carefully aspirated and washed with PBS twice. Afterwards the DNA was digested using 0.02 mg/ml DNAse I (Worthington) in PBS with calcium and magnesium for 1 h at 37° C. Finally, the MSC-derived matrices were washed twice with PBS at room temperature and stored in PBS at 4° C. for up to two months. The decellularized customized cell-derived ECM presented itself as a network of thick and thinner fibrils with a heterogeneous mesh size equally distributed over the culture surface, free of cellular components.

### Example 2

After 2 days of culture, MSCs' culture medium was aspirated and the cell layers were stored at −80° C. until use. mRNA was purified using RNAiso Plus (cat# 9109, Takara) by following the manufacturer's instructions for cells grown in monolayers. The mRNA concentration was assessed using a nanodrop and then converted to complementary DNA (cDNA) by using reverse transcriptase (PrimeScript RT Master Mix, cat.# RR036A; Takara) and following the respective user manual. The cDNA product was stored at −20° C. and used for further amplification of the desired gene sequences. The primer sequences utilized for amplification of human IL10 were:

Forward
(SEQ ID NO: 1)
5′-TCAAGGCGCATGTGAACTCC-3′;
Reverse
(SEQ ID NO: 2)
5′-GATGTCAAACTCACTCATGGCT-3′

and for human GAPDH were:

Forward:
(SEQ ID NO: 3)
5′-CCAGGGCTGCTTTTAACTCTGGTAAAGTGG-3′;
Reverse:
(SEQ ID NO: 4)
5′-ATTTCCATTGATGACAAGCTTCCCGTTCTC-3′.

cDNA amplification and respective quantification of the target sequences was achieved with TB Green Premix Ex Taq (cat.# RR420A; Takara) by following manufacturer's instructions. The obtained Ct values for IL10 were normalized to GAPDH Ct values and expressed as fold-change to non-induced MSC-IL10 normalized values.

HMWHA and MMC promoted an anti-inflammatory phenotype in MSCs, as evident by a 2- to 4-fold increase in IL10 mRNA expression. The combination of both HMWHA and MMC had an orthogonal effect, inducing a 17-fold increase in IL10 mRNA expression in MSCs. This response largely surpasses IL10 expression of HMWHA and MMC cultures alone.

### Example 3

Human THP-1 cells (ATCC) were differentiated into macrophages by seeding them on 0.1% gelatin coated TCP at 100,000 cells/cm² in growth medium (Roswell Park Memorial Institute 1640, RPMI 1640, with 10% FBS and 1% P/S) containing 100 ng/ml of phorbol 12-myristate-13-acetate (PMA) overnight. The cell layer was then trypsinized with trypLE for 6 minutes at 37° C. and seeded with growth medium on the desired substrate at 20,000 cells/cm². Attachment and resting took place for 24 h. Macrophages were then washed with PBS and polarized with 10 ng/ml LPS and 5 ng/ml IFNγ in 5% FBS medium (RPMI 1640 with 5% FBS and 1% P/S) for 30 minutes at 37° C. The cell layer was washed with PBS and allowed to condition new 5% FBS medium for 24 h. The conditioned medium was then collected for ELISA and stored at −80° C. ELISA was performed according to manufacturer's protocol (PeproTech). It was found that ECMs assembled in the presence of HMWHA, MMC alone or in the presence of the combination of both, completely blocked polarization towards M1 phenotype. Secreted TNFα levels in culture media from macrophages cultured on these matrices were equivalent to non-polarized controls. These results were specific for our customized matrices, as macrophages on unspecific ECM coating (gelatin), as well as on TCP showed a strong pro-inflammatory response (high TNFα levels). Control ECM derived from MSCs cultured without HMWHA and MMC was only able to buffer naïve macrophage-to-M1 polarization by 50%. Hence, it was shown that MSCs, when exposed to HMWHA and MMC deposit a strongly anti-inflammatory ECM, which can even inhibit M1 polarization of macrophages.

### Example 4

Human umbilical cord endothelial cells (HUVECs) were cultured between passage 4 and 8 and used to form spheroids (˜700 cells/spheroid) in low adhesion microwells. The spheroids were embedded in a collagen I hydrogel (1 mg/ml) and seeded on top of TCP unmodified MSC-derived ECM (cECM) or DxS-ECM (MSC-derived ECM deposited in the presence of dextran sulfate (DxS, 500 KDa, 10 μg/ml)). Spheroids were cultured on ECM-based biomaterials or TCP for 24 h, followed by 4% PFA fixation and staining of actin filaments with Phalloidin for better visualization of the cell shape. Measurement of the cumulative length of the endothelial sprouts showed that MSC-derived ECMs significantly increased spheroid sprout length in relation to TCP. This pro-angiogenic potential of unmodified MSC-derived ECM was further exceeded by the superior pro-angiogenic activity of DxS-ECM, as significantly longer sprouts were observed.

### Summary

During life many tissues face injury or degeneration, due to trauma, aging, disease or simply wear-and-tear. Examples for these kinds of situations include skin cuts, bone fracture, sarcopenia, osteoarthritis, liver cirrhosis, ischemic diseases such as chronic wounds, myocardial infarction and stroke, just to name a few. Such tissues are required to heal and regenerate to fulfil their essential function in the body⁴¹.

Unfortunately, some tissues have a very limited ability to heal and regenerate. This process is further impaired by a chronically inflamed and dysregulated microenvironment. Examples for such non-healing and degenerating tissues include diabetic chronic wounds, myocardial infarction and osteoarthritis, just to name a few examples²⁻⁵.

During normal and functional wound healing, upon injury the damaged tissue and necrotic cells initiate an inflammatory response, which is necessary to clear debris, recruit cells and initiate the healing cascade. This acute inflammatory response is followed by a proliferative phase in which endothelial cells form new blood vessels (angiogenesis) and tissue forming cells (e.g., fibroblasts) deposit new ECM, thereby forming de novo tissue. This is followed by a remodeling phase, during which the de novo tissue (regeneration) or the scar (healing) matures. Hence, in order for healing to progress, the acute inflammatory response has to be down-regulated after a short peak and the damaged tissue area is required to revascularize for other cells to form and remodel new tissue⁴³.

In many tissues with a limited regeneration potential, such as myocardium, or under diseased conditions (e.g., diabetes), this healing cascade is dysregulated, resulting in a chronic inflammatory response and ischemia^{21,33,39,42}. Chronic inflammation and ischemia does not only impair healing and regeneration, but also negatively affects the surrounding tissue, putting it at risk. In particular, inflammatory factors, proteases and reactive oxygen species from the chronically inflamed tissue and lack of sufficient oxygen also damage the surrounding tissue, resulting in an expansion of the tissue damage and thus further loss of function^{21,33,39,42}.

This can have fatal effects, for example, when a myocardial infarct reaches a critical size leading to chronic heart failure. It can also necessitate amputation, e.g., in the case of diabetic chronic wounds^{21,33,39,42}.

Hence, the chronically inflamed, ischemic and dysregulated microenvironment in non-regenerating and non-healing tissues is a major therapeutic target. Since an inflammatory response is essential during wound healing and regeneration, it cannot be broadly down-regulated or “switched-off”²¹. Such approaches were previously demonstrated to completely halt the healing response²¹. Instead, the hostile dysregulated chronically inflamed and ischemic microenvironment has to be modulated and turned into a pro-healing one. In order to achieve this, complex biological processes have to be finely tuned and adjusted⁴⁴.

One of the detrimental cell types in the whole healing process are macrophages. These exhibit a wide spectrum of phenotypes in between two extrema, M1 and M2. Pro-inflammatory macrophages (M1) are predominantly present in the inflammatory phase, whereas anti-inflammatory and wound healing macrophages (M2) are accumulating in the reparative phase. Macrophages communicate with cells from the innate and adaptive immune system, regulate ECM remodeling, angiogenesis and fibrosis, and thus are one of the major cell types responsible for the healing outcome⁴³. Importantly, a prolonged presence of inflammatory (M1) macrophages leads to an extensive chronic inflammatory phase that negatively impacts healing progression and the viable cells at border zone⁴³. Therefore, macrophages represent a promising therapeutic target to counteract chronic inflammation⁴¹.

The present inventors have developed a bio-instructive biomaterial based on customized cell-derived extracellular matrix (extracellular matrix material), engineered to modulate inflammatory responses and be generated in sufficient amounts with a stable and reproducible bioactivity. In particular, this extracellular matrix material is able to completely block the polarization of macrophages towards a pro-inflammatory M1 phenotype.

Additionally, dysregulated tissue microenvironments are also often characterized by an ischemic microenvironment that due to limited blood (thus oxygen and nutrient) supply delays or prevents healing. Hence, the delivery of pro-angiogenic factors was thought as a promising approach to promote healing in ischemic tissues. However, the delivery of angiogenic growth factors fails to succeed in vivo, as they have very short life-time on their own⁴⁵. Additionally, there are difficulties in translating growth factor-based technologies due to the immense side-effected caused by necessary supra-physiological doses.

An extracellular matrix of human origin is capable of addressing these limitations since angiogenic factors are naturally incorporated in the ECM as they are secreted, where they remain stable³⁷. Some ECMs disclosed in this invention efficiently proved this concept by showing superior pro-angiogenic properties in a spheroid sprouting assay.

The extracellular matrix material can be collected and stored under cold temperature and therefore used off-the-shelf. It can be processed and incorporated in all types of materials, including tissue scaffolds, implants, wound dressings and (injectable) hydrogels. Thus, just by itself or incorporated into other materials, the extracellular matrix material can be applied to tissue areas with chronically inflamed and dysregulated microenvironments, thereby modulating and turning the diseased environment into a pre-healing one. This will advance the healing and regeneration process in non-healing and non-regenerative tissues, such as chronic diabetic wounds, infarcted myocardium and osteoarthritis.

### Introduction

Various experimental approaches exist that attempted to improve the hostile chronically inflamed or ischemic microenvironment in various diseases. These include growth factor and gene therapy, cell-based therapy, tissue-derived ECM, and various bioengineered scaffolds mimicking isolated properties of the ECM.

Especially, MSCs were believed to be very promising due to their immunomodulatory and anti-inflammatory and pro-angiogenic properties^{16,44,45}. Unfortunately, the hostile microenvironment severely limits engraftment and survival, thus impairing the regenerative effect of MSCs.

Nevertheless, MSCs are ascribed strong microenvironment-improving abilities⁴⁶. Various soluble factors and extracellular vesicles (exosomes) secreted by MSCs have been identified to be in part responsible for their mechanism of action. As a result, more recent approaches enriched these secreted components to be applied into the affected area^{44,45}. MSCs are stromal cells, thus are also competent insoluble-ECM producers. Yet, MSC-derived ECM thus far has not been investigated for its ability to promote tissue repair in dysregulated inflamed tissues.

The ECM is a biomaterial designed by nature, which has undergone more than 500 million years of material optimization. It signals cells using a combination of three major communication planes (biochemical composition, biomechanical properties and topography)¹². In a physiological connective tissue environment, the ECM is known to bind, sequester, preserve, present and modulate the activity of signaling molecules, including cytokines, also found in the bioactive soluble fraction of the MSCs' secretome. The accurate organization of these signalling components is a prerequisite to harness their full bioactive strength and ensure long-term stability^{12,13.} Hence, this complexity in communication allows the ECM to orchestrate processes such as tissue healing and regeneration¹².

Beneficial effects of ECM derived from tissues, such as dermis and myocardium, were already demonstrated to in various experimental models for various diseases¹⁴⁻¹⁷. Nonetheless, tissue-derived ECM faces many limitations in clinical application, such as risk of disease transmission, limited availability of human tissue, immunological rejection of animal-derived products and the lack of bioactivity customization. Its complexity and fixed composition confounds our understanding of the mechanism of action, thus diminishes the predictability of the ECM's therapeutic effect¹⁸⁻²². In view of the above, instead of utilizing tissue-derived ECM or transplanting MSCs, the present inventors have customized MSC-derived ECM to modulate the hostile environment in dysregulated chronically inflamed and ischemic tissue microenvironments.

### Background

In vitro ECM: MSC-derived ECMs were shown to be cell-rejuvenating²³, promote peripheral nerve growth in vitro²⁴ and even recapitulate the bone marrow niche sufficiently to expand hematopoietic progenitor cells without decreasing their long-term engraftment ability²⁵.

The majority of previous studies on in vitro cell-derived ECM, however, focused on osteoblast- or chondrocyte-derived in vitro ECMs and demonstrated that these ECMs were not sufficient to induce terminal differentiation on their own. Nonetheless, they strongly augmented the differentiation of stem cells induced by standard differentiation factors^{26,27}. In vivo, several studies showed osteogenic potential of osteoblast-derived ECM^{28,29}, whereas in other studies such effects were not observed³″¹. Hence, although lineage specific in vitro ECM can be generated, the strength of its bioactivity is not guaranteed by current standard culture methods.

A major limitation of in vitro ECMs is their instable bioactivity, caused by too little amounts of ECM that are deposited under standard culture and further decreased after decellularization³².

Macromolecular crowding: Previously MMC was used as a biophysical principle in in vitro biological systems, see, e.g., U.S. Pat. No. 9,809,798, WO2011108993A1, WO2015187098A1, and WO2014077778 A1. In tissue, the cellular exterior is cramped with macromolecules. In order to emulate the crowded in vivo conditions, macromolecules were incorporated into the cultures, which occupy space and thereby increase the effective concentration of all components secreted into the biological system. The change in the relationship between total volume and available volume (Vₜₒₜₐₗ/V_{available}>1) increased the thermodynamic activity within cell culture system and resulted in increased reaction kinetics including enzyme kinetics and amplified molecular interactions³³. Successful application of this biophysical principle by accelerating enzyme kinetics such as procollagen C protease has been demonstrated, leading to enhanced collagen I deposition³³ and collagenase activity³⁴ under MMC.

It has also been shown that MMC increases supramolecular assemblies¹³, ECM cross-linking and stabilization¹¹, as well as ECM remodelling ^{11,12.} Under MMC the amount of deposited ECM after a few days exceeds the amount of ECM, which can be accumulated within weeks under standard culture conditions, several fold¹¹.

It has been also shown recently that some macromolecules enhance ECM deposition independent of an MMC effect, but rather by aggregating and co-precipitating with the assembled ECM¹⁴.

In vitro-derived ECM generated under MMC was shown to drive terminal differentiation of MSCs into adipocytes without the addition of any inductive factors. This is in contrast to state-of-the-art studies utilizing cell-derived ECM generated without MMC^{26,27} and the no-MMC ECM controls³⁴.

MMC effects in cell culture are not restricted to ECM formation. It has been shown that MMC could enhance proliferation in various cell types¹¹ and enabled the sourcing of hematopoietic pericytes from human peripheral blood^{15,16}. The effect of MMC on the anti-inflammatory properties of cells such as MSCs and their respective ECM or the pro-angiogenic properties of the ECM is yet to be investigated.

Pre-conditioning of MSCs towards an anti-inflammatory phenotype: In general, pre-conditioning of MSCs activates their immunomodulatory and anti-inflammatory properties. These include pre-treatment with hypoxia or pro-inflammatory factors such as IFNγ¹⁶, LPS or IL1β¹⁷. Nonetheless, such pre-treatments have their own limitations, as accidental co-delivery of these pro-inflammatory factors might have adverse effects. In addition, over-exposure of MSCs to the pro-inflammatory molecule LPS was shown to induce a pro-inflammatory phenotype¹⁷.

### This Invention

MSCs were conditioned with HMWHA while promoting MSC-derived ECM deposition by MMC using our established neutral crowder cocktail based on Ficoll 70 kDa and 400 kDa^{11,12,14}. Hyaluronic acid was chosen as it resembles one of the fundamental ECM components in tissue development, regeneration and repair⁵². It is indispensable for scarless regeneration in mammalian fetal skin wounds⁵³ and in the zebrafish heart ⁵⁴. HMWHA was demonstrated to be anti-inflammatory, immunomodulatory and anti-oxidant⁵⁵.

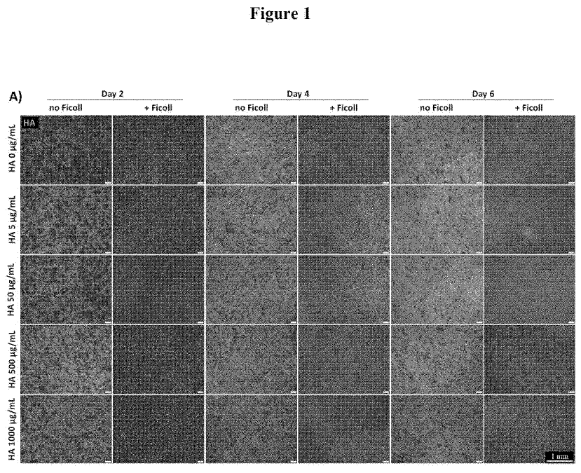

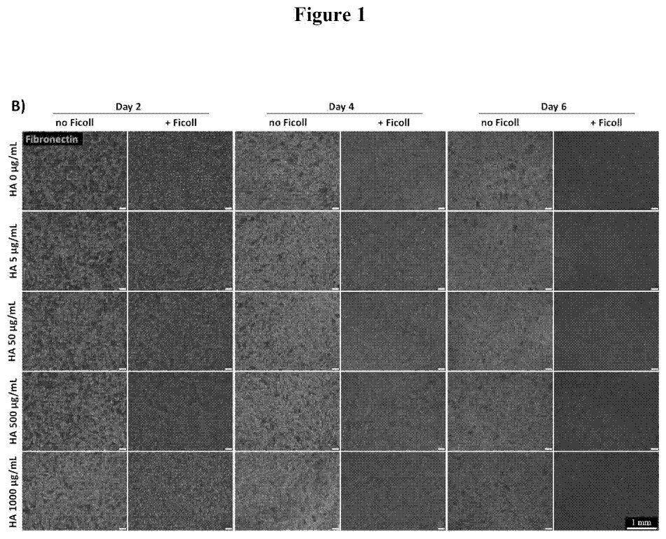

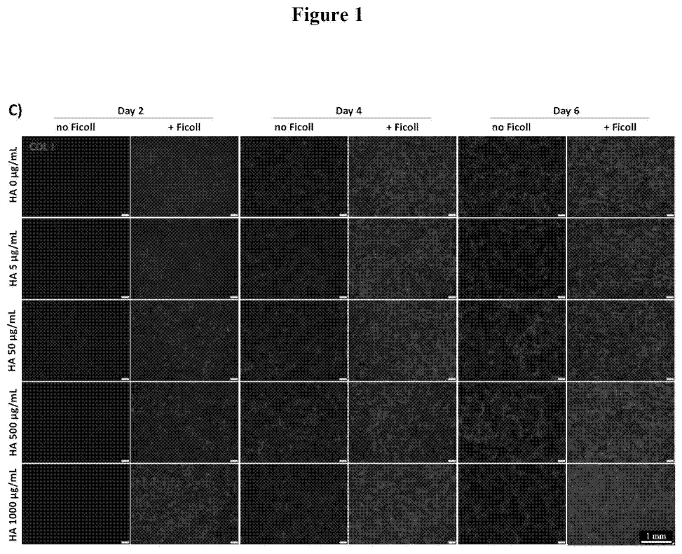

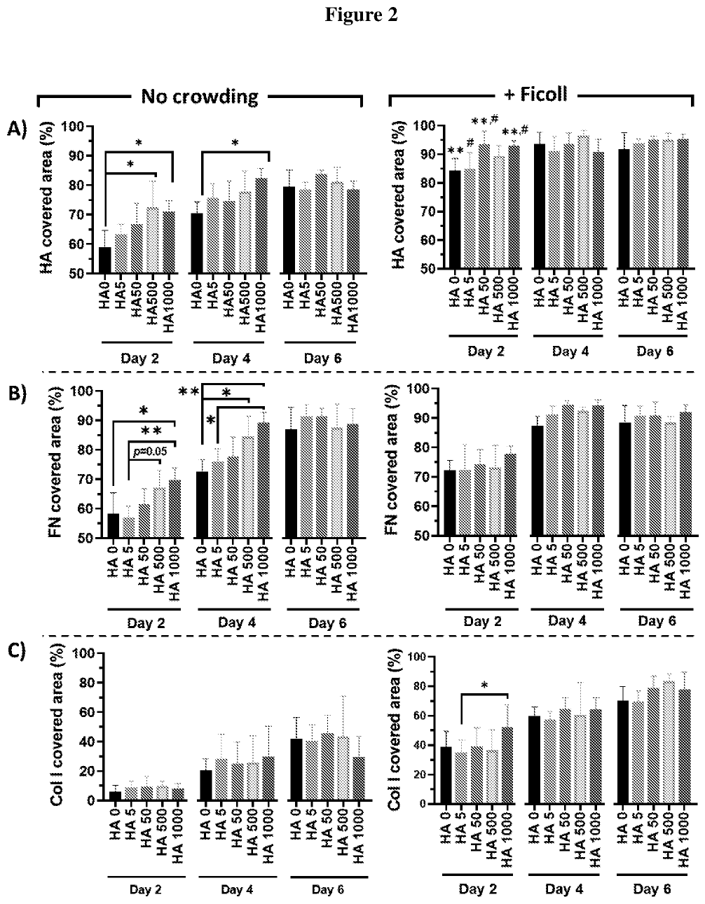

Bone marrow MSCs cultured under standard conditions (no exogenously added HMWHA and no MMC) already assembled an ECM rich in HA and fibronectin with a dense fibrillar pattern (FIG. 1). Both HA and fibronectin seem to be deposited early at a similar rate, while deposited collagen I was only detectable from day 4 onwards. To investigate the effect of HMWHA on ECM deposition, bone marrow MSCs were cultured in the presence of exogenously added HMWHA at a concentration ranging from 5 and 1000 μg/ml. Supplementation of HMWHA led to the progressive enrichment of the hyaluronic acid and fibronectin in the cell-derived ECM in a HMWHA-dose dependent manner (FIGS. 1 A,B and 2 A,B). No significant effect of supplemented HMWHA on collagen I deposition was observed (FIGS. 1C and 2C).

Next, the established neutral MMC cocktail was also supplemented based on ficoll 70 kDa (37.5 mg/ml) and Ficoll 400 kDa (25 mg/ml) to the MSC cultures. It was observed that MMC drove deposition of all ECM components, already reaching full surface area coverage for hyaluronic acid and fibronectin on day 4 and significantly increasing collagen I deposition (FIGS. 1 and 2). Importantly, co-supplementation of HMWHA and MMC did not lead to any adverse effects on ECM deposition. Instead MMC's strength to drive ECM deposition masked that of HMWHA for all time points and ECM components. The exception to that was observed on day 2, when a HMWHA-dose dependent increase of assembled hyaluronic acid and collagen I was still detected under MMC.

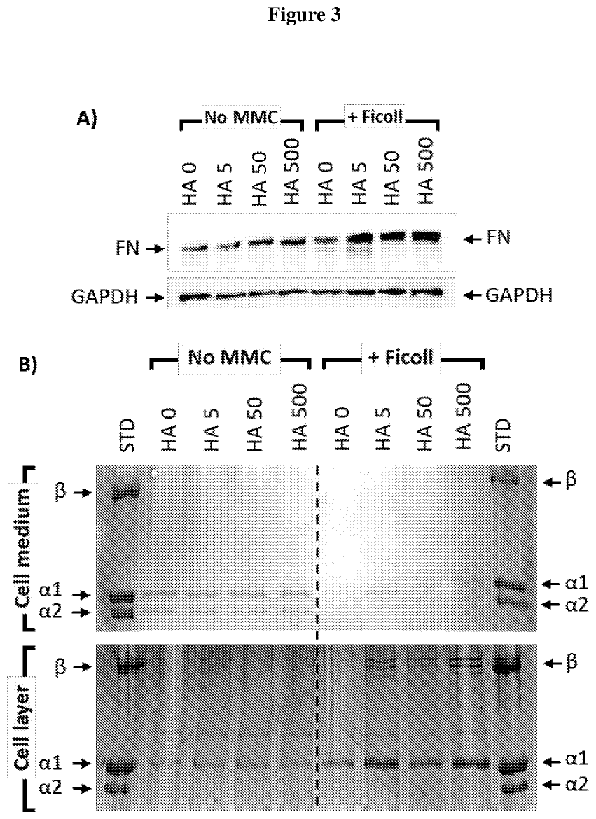

Western blot analysis of total protein extracts from the respective cell layers on day 6 confirmed the above stated trends in fibronectin deposition, depicting a strongly enhanced ECM deposition under MMC (FIG. 3A).

Collagen deposition was further investigated by digesting culture media (supernatant) and cell layer samples with pepsin after 6 days of culture and then visualizing the remaining non-digested collagenous bands on a silver-stained SDS-PAGE gel (FIG. 3B). As it was demonstrated in FIG. 1 that collagen I deposition did not significantly increase with HMWHA 1000 μg/ml at day 6, these experiments were performed with MSCs incubated with 5-500 μg/ml of HMWHA, with or without MMC. Collagen I α1 and α2 chains were clearly detectable in the cell culture media of all non-crowded samples, while no collagen I was detectable in samples supplemented with MMC (FIG. 3B). No significant differences were observed between samples containing different concentrations of HMWHA. In conjunction, highest amounts of deposited collagen I were detected in the cell layer of samples supplemented with MMC. Nonetheless small amounts of collagen I were also observed in non-crowded samples (FIG. 3B). These data confirm the trend observed for collagen I in the immuno-stained samples (FIGS. 1C and 2C).

As HMWHA (500 μg/ml) samples showed the best ECM deposition in comparison to their respective no-HMWHA samples, we decided to proceed only with HMWHA (500 μg/ml). This concentration of HMWHA was used for further experiments to evaluate cellular responses of MSCs directly to HMWHA and/or MMC and macrophage responses to the ECMs derived under the respective conditions.

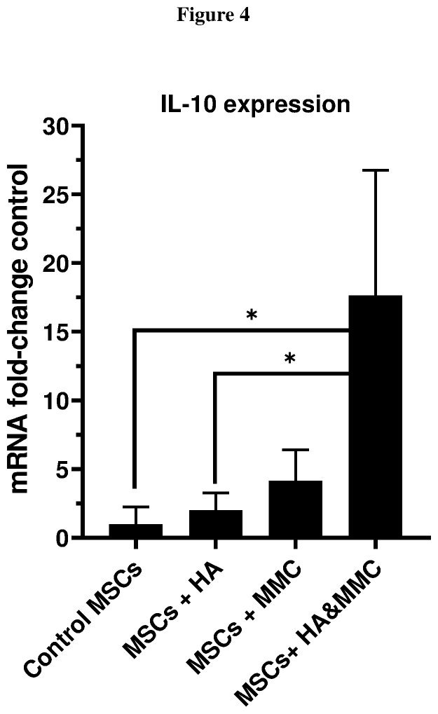

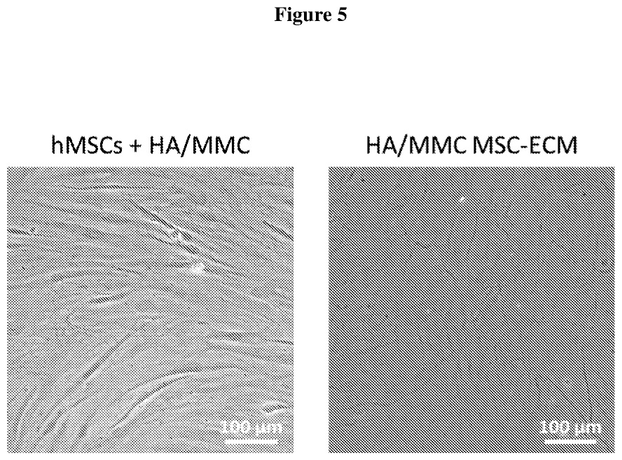

Hence, the anti-inflammatory properties of MSCs cultured in the presence of HMWHA (500 μg/ml) and/or MMC were investigated. After 2 days of culture, levels of IL10 mRNA expression were quantified (FIG. 4). Strikingly, it was discovered that both HMWHA and MMC promoted an anti-inflammatory phenotype in MSCs, as evident by a 2- to 4-fold increase in IL10 mRNA expression. IL10 is one of the major paracrine factors involved in the anti-inflammatory effect of MSCs¹⁶. This finding was not obvious as until now only pre-treatment with pro-inflammatory factors was demonstrated to enhance an anti-inflammatory phenotype^{16,17,} whereas HMWHA is known to be strongly anti-inflammatory⁵⁵. Further, there was no prior indication that MMC would condition MSCs towards an anti-inflammatory phenotype. Even more surprising, the combination of both HMWHA and MMC had an orthogonal effect inducing a 17-fold increase in IL10 mRNA expression in MSCs. This response largely surpasses IL10 expression of HMWHA and MMC supplemented cultures alone.

The matrices were decellularized using sodium deoxycholate (DOC) in combination with DNase. This method resulted in the best preservation of ECM components (see fibrillar structures), while all cells and their genomic content were removed (FIG. 4; see also Materials and Methods). This is essential for preservation of the bioactive components and low immunogenicity^{48,50,56}.

The decellularized extracellular matrix material presented itself as a network of thick and thinner fibrils with a heterogeneous mesh size equally distributed over the culture surface. This extracellular matrix material was mechanically resistant to the decellularization method, hence increasing reproducibility.

Macrophages, differentiated from THP-1 human lymphocytic cell line, were used to test the bioactivity of the extracellular matrix material. These macrophages are able to polarize towards a pro-inflammatory (M1) or an anti-inflammatory (M2) phenotype, given pro- or anti-inflammatory stimuli, respectively⁵⁷.

In order to verify that the anti-inflammatory phenotype of MSCs induced by HMWHA and/or MMC (FIG. 4) would be also reflected in the bioactivity of the respective extracellular matrix material, the ability of the extracellular matrix material to act as a potent anti-inflammatory microenvironment to inhibit inflammation was investigated.

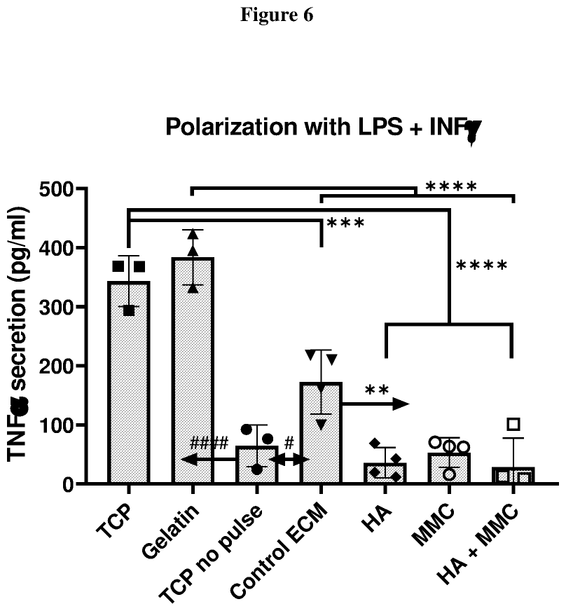

THP-1 cells were differentiated into macrophages overnight, then seeded on the extracellular matrix material and allowed to attach for another day. Next, macrophages were polarized towards a pro-inflammatory M1 phenotype by pulsing with LPS and IFNγ. The macrophages were allowed to condition fresh medium with their secreted factors for 24 hours, after which the supernatant was analysed for secreted amounts of pro-inflammatory TNFα by ELISA (FIG. 6). It was discovered that ECMs under HMWHA or MMC alone and HA together with MMC completely inhibited macrophage polarization towards a M1 phenotype. Secreted TNFα levels in culture media from macrophages cultured on these matrices were equivalent to non-polarized controls. These results were specific for the engineered matrices, as macrophages on control MSC-derived ECM and unspecific ECM coating (gelatin), as well as on TCP, showed a strong pro-inflammatory response (high TNFα levels) (FIG. 6).

It is shown for the first time that MSCs, when exposed to HMWHA and/or MMC, deposit a strongly anti-inflammatory ECM, which can even inhibit M1 polarization of macrophages. These findings are not obvious, as cell-derived ECM of any source (including MSCs) has not been investigated for its anti-inflammatory properties before. Further, it has not been shown previously that HMWHA and MMC can enhance the anti-inflammatory phenotype of MSCs, including the anti-inflammatory properties of the extracellular matrix material. As the levels of HMWHA were comparable between all conditions on day 6 under MMC, enhanced anti-inflammatory properties of extracellular matrix material cannot be attributed to higher levels of HMWHA.

Hence, the method described herein uses HMWHA, MMC or the combination of both to deposit an anti-inflammatory ECM, which can be harvested, optionally further processed and applied to modulate a chronically inflamed dysregulated tissue microenvironment.

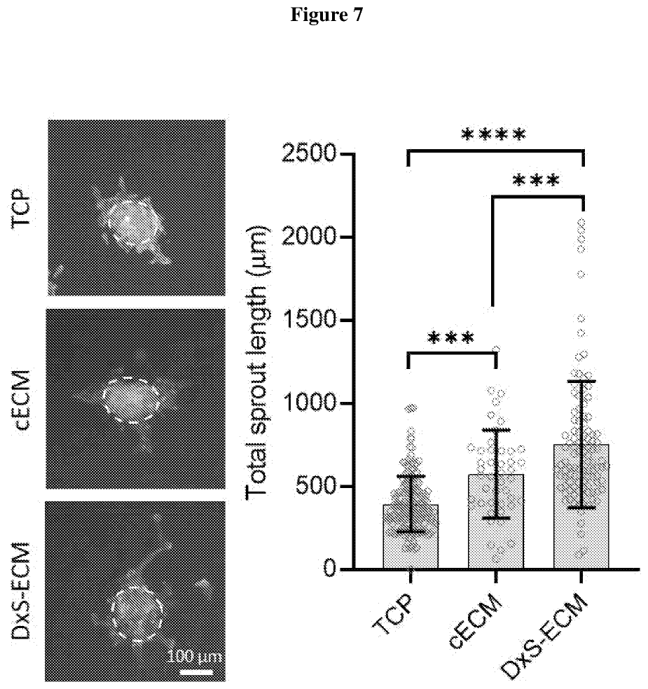

DxS was also used to supplement MSCs cultures. It has previously been shown that addition of DxS leads to a significant enhancement in ECM deposition in MSC cultures by aggregation and co-precipitation of MSC-derived ECM with DxS¹⁴. MSC-derived ECMs assembled in the presences of DxS (500 kDa, 10 μg/ml) were decellularized . This DxS-ECM was used as substrate for a culture of endothelial spheroids embedded in a collagen I hydrogel (FIG. 7). Unmodified control MSC-ECM (produced in the absence of DxS) was also tested and TCP was used as a no-ECM control. After 24h the endothelial spheroids have formed vessel sprouts. Quantification of the cumulative sprout length per spheroid showed a significant increase to TCP as well as the unmodified MSC-derived ECM.

Hence, deposition of MSC-derived ECM in the presence of DxS resulted in an ECM-based biomaterial with superior pro-angiogenic properties.

According to these observations, the invention is also directed to the generated ECM (extracellular matrix material), which can be harvested, stored, further processed and applied to modulate a chronically inflamed and/or ischemic dysregulated tissue microenvironment.

### Materials and Methods

### HMWHA and Ficoll70/400 Preparation

HMWHA (1.5-1.8MDa) was purchased from Sigma Aldrich and diluted to 2 mg/ml in DMEM (Gibco) with 1 g/L glucose supplemented with GlutaMAX. Complete dissolution was achieved with agitation at room temperature for 6-8 h. The prepared solution was filtered to sterility and stored at −20° C. for a maximum of 6 months and freeze-thaw cycles were avoided.

Ficoll 70 kDa (75 mg/ml) (GE Healthcare) was mixed with ficoll 400 kDa (50 mg/ml) (GE Healthcare) and dissolved in DMEM with 1 g/L glucose and GlutaMAX. Agitation for 30 minutes at room temperature ensured total dissolution. The ficoll70/400 solution (MMC) was filtered to sterility and used on the same day.

Dextran sulfate (500 kDa, 10 mg/ml) (Sigma Aldrich) was dissolved in water and filtered to sterility to achieve a 1000-times stock. DxS was diluted 1:1000 in DMEM with 1 g/L glucose and GlutaMAX additionally supplemented with 0.5% FBS and 0.1 mM ascobic acid (Sigma-Aldrich)

### MSC Culture

Human bone marrow MSCs were obtained from different donors (Millipore; Lonza) and cultured individually as follows. MSCs were seeded at 4-6,000 cells per cm² in TCP coated with 0.1% gelatin and expanded using DMEM with 1 g/L glucose supplemented with GlutaMAX and additional 10% FBS (Gibco) and 100 U/ml penincilin and 100 μg/ml streptomycin (1% P/S) at 37° C. in 5% CO₂. MSCs were then trypsinized with TrypLE (Gibco) and seeded between passage 6 and 9 at 6,500 cells per cm² in TCP plates at 0.3 ml volume per cm². The cells were allowed to attach for 24 h in DMEM with 10% FBS and 1% P/S, after which the medium was exchanged for induction medium used to promote ECM assembly.

### MSC Induction to Promote ECM Assembly

Induction of MSCs was done using mixtures of 1 part freshly made ficoll70/400 and 1 part of DMEM or HMWHA diluted in DMEM to the desired final concentration (0-1000 μg/ml). This medium was additionally supplemented with 0.5% FBS and 0.1 mM ascobic acid (Sigma-Aldrich). Alternatively, MSCs were exposed to media composed of DxS (500 kDa, 10 μg/ml) in DMEM with 1 g/L glucose and GlutaMAX additionally supplemented with 0.5% FBS and 0.1 mM ascobic acid (Sigma-Aldrich). Control induction medium was comprised of DMEM 0.5% FBS and 0.1mM ascorbic acid only. Cells were cultured for a maximum of 6 days without medium change and were then prepared for further analysis or processing.

### Decellularization of MSC-derived ECM

After 6 days of culture MSCs were carefully washed with PBS twice at room temperature. Plates were placed on ice and washed with 0.5% sodium deoxycholate (DOC in water; Sigma) containing 0.5× protease inhibitor (from 400× stock in dimethyl sulfoxide) for 15 minutes. This solution was then replaced by 0.5% DOC in water for 10 minutes at room temperature. The solution was then carefully aspirated and washed with PBS twice. Afterwards the DNA was digested using 0.02mg/ml DNAse I (Worthington) in PBS with calcium and magnesium for 1 hour at 37° C. Finally, the MSC-derived matrices were washed twice with PBS at room temperature and stored in PBS at 4° C. for up to two months.

### THP-1 Culture, Differentiation and Subsequent Polarization

THP-1 cells (ATCC) were cultured between 10,000 and 1 million cells per milliliter in growth medium (RPMI 1640 with 10% FBS and 1% P/S). The cells were seeded 0.1% gelatin coated TCP at 100,000 cells/cm² in growth medium containing 100 ng/ml of PMA. THP-1 differentiated overnight and were attached afterwards. The cell layer was then trypsinized with trypLE for 6 minutes at 37° C. and seeded with growth medium on the desired substrate (control ECM, HMWHA, MMC, HMWHA with MMC, TCP, gelatin 1%) at 20,000 cells/cm². Attachment and resting took place for 24h. Macrophages were then washed with PBS and polarized with 10 ng/ml LPS (Sigma) and 5 ng/ml IFNy (PeproTech) in 5% FBS medium (RPMI 1640 with 5% FBS and 1% P/S) for 30 minutes at 37° C. The cell layer was washed with PBS and allowed to condition new 5% FBS medium for 24 h. The conditioned medium was then collected for ELISA and stored at −80° C. ELISA for TNFα was performed according to manufacturer's protocol (PeproTech).

### Endothelial Cell Sprouting Assay

Human umbilical vein endothelial cells (HUVECs, ATCC, pooled donors) were seeded at 2.5-5,000 cells/cm², in TCP coated with 0.1% gelatin, and expanded in endothelial cell growth medium formulation 2 (EGM2, Lonza) until 80% confluency. HUVECs were then trypsinized with TrypLE (Gibco) and seeded between passage 4 and 8 in low adhesion microwells at 700 cells per microwell. The cells were allowed to form spheroids overnight and the resulting spheroids were then collected and diluted in a collagen I hydrogel solution (1 mg/ml) made with EGM2. The spheroid-containing collagen I solution was added to MSC-derived ECM deposited in the presence of DxS, to unmodified MSC-derived ECM coated plates or ECM-free bare TCP plates and allowed to polymerize for 2 h at 37° C. The hydrogels were then overlayed with EGM2 and the spheroids were allowed to sprout for 24 h, after which they were fixed with 4% PFA and stained with Phalloidin-alexa fluor 555 (abcam) for detecting filamentous actin (F-actin). F-actin was used to determine cell shape and position, which was used to quantify the cumulative sprout length of endothelial cell spheroids using Image J v1.52i software.

### RT-qPCR for Detection of Inflammatory-cytokine Expression

After 2 days of culture of MSCs culture medium was aspirated and the cell layers were stored at −80° C. until use. mRNA was purified using RNAiso Plus (cat# 9109, Takara) by following the manufacturer's instructions for cells grown in monolayers. The mRNA concentration was assessed using a nanodrop and then converted to cDNA by using reverse transcriptase (PrimeScript RT Master Mix, cat.# RR036A; Takara) and following the respective user manual. The cDNA product was stored at −20° C. and used for further amplification of the desired gene sequences.

The primer sequences utilized for amplification of human IL10 were forward: 5′-TCAAGGCGCATGTGAACTCC-3′ (SEQ ID NO:1) and reverse:

5′-GATGTCAAACTCACTCATGGCT-3′ (SEQ ID NO:2); and for human GAPDH were forward: 5′-CCAGGGCTGCTTTTAACTCTGGTAAAGTGG-3′ (SEQ ID NO:3) and reverse: 5′-ATTTCCATTGATGACAAGCTTCCCGTTCTC-3′ (SEQ ID NO:4). cDNA amplification and respective quantification of the target sequences was achieved with TB Green Premix Ex Taq (cat.# RR420A; Takara) by following manufacturer's instructions. The obtained Ct values for IL10 were normalized to GAPDH Ct values and expressed as fold-change to non-induced MSC IL10 normalized values.

### Immunocytochemistry

Cell layers were washed with PBS and fixed for 10 min with ice cold methanol. Subsequently, cell layers were blocked with 3% bovine serum albumin (BSA) for 1 h and cell layers were incubated overnight at 4° C. with the primary antibodies in 1% BSA in PBS. Next, secondary antibodies or other dyes were added for 2 h at room temperature. Finally, the samples were washed with PBS and visualized. The following primary antibodies and reagents against human antigens were obtained from abcam (Hong Kong, HK SAR): polyclonal to hyaluronic acid (1:500; cat. #ab53842), polyclonal to fibronectin (1:500 for cytochemistry and 1:6,000 for Western blot; cat. #ab2413) and monoclonal to GAPDH (1:6,000; cat. #ab181602). Antibody to human collagen I was used at 1:1000 (cat. # C2456, Sigma-Adrich, Saint Louis, USA). Secondary antibodies used comprise abcam Alexa Fluor 488 (1:1,000; cat. #ab150077), Alexa Fluor 555 (1:500; cat. #ab150178) and Alexa Fluor 594 (1:500; cat. #ab150160). Alexa Fluor 647 (Molecular Probes, Life Technologies Grand Island, N.Y., USA; cat. #A31571) and 4′, 6-diamidino-2-phenylindole (DAPI; BD Pharmingen, San Diego, Calif., USA; cat. #564907) were used at 1:1,000. An horseradish peroxidase (HRP)-conjugated antibody was kindly provided by Thermo Fisher Scientific (1:5,000; Rockford, Ill., USA; cat. #A27036). Reagents and instruments for electrophoresis and Western blots were purchased from Invitrogen (Life Technologies, Rockford, Ill., USA).

### Western Blot

The cell layers were washed with PBS and lysed with 1 part of sample buffer (0.25

M Tris pH 6.8, 4% SDS and 20% Glycerol) and 1 part of 2X protease inhibitor cocktail (Sigma-Aldrich). Lysates were denatured at 95° C. with 10% 2-mercaptoethanol and resolved by SDS-PAGE. The gel was transferred to a polyvinylidene difluoride membrane and detected by western blot using ECL Super Signal West Pico Plus (Life Technologies).

### Pepsin Digestion, SDS-PAGE and Silver Staining

The cell culture medium was collected and the cell layer washed with PBS. One part of culture medium was digested with 1 part of 1 mg/ml pepsin (cat.#V195A, Madison, Wis., USA) in 1N HCl, while the cell layer was diggested with 60 μl/cm² of 0.25 mg/ml pepsin-0.5% Triton-X-100 (Sigma, Saint Louis, USA) in 0.25 N HCl. The digestion was carried out for 3 hours under agitation and the reaction was stopped by adding 1N NaOH in proportion to the N of HCl in the reaction. The extracts from the cell layer were collected and analysed together with the respective cell medium extracts by SDS-PAGE. Briefly, the samples were diluted 1:1 in sample buffer (0.25 M Tris pH 6.8, 4% SDS and 20% Glycerol), resolved by SDS-PAGE and the gels were stained using Silver Staining Plus kit (cat.# 161-0449, Bio-Rad laboratories, Inc., USA).

### Microscopy

Studies were performed using a Olympus IX83 inverted fluorescence microscope suited with CellSense Dimention image acquisition software. Images were processed and quantified using Image J v1.52i software (website: imagej.nih.gov/ij/).

### Statistical Analysis

Statistical analysis was performed after confirming the assumptions of normality and equal variance were met. Two-way Analysis of Variance algorithm and post-hoc Tukey tests were used and p-values bellow 0.05 were considered statistically significant. The analysis was performed using GraphPad Prism v8.0 (GraphPad Software, San Diego, Calif., USA, website: graphpad.com).

All patents, patent applications, and other publications, including GenBank Accession Numbers, cited in this application are incorporated by reference in the entirety for all purposes.

### LIST OF REFERENCES

### 1. Atala, A., Irvine, D. J., Moses, M. & Shaunak, S. MRS Bull. 35, 597-606 (2010).

### 2. Zhang, W., Ouyang, H., Dass, C. R. & Xu, J. Bone Res. 4, 15040 (2016).

3. Frangogiannis, N. G. Antioxid. Redox Signal. 8, 1907-1939 (2006).
4. Ong, S.-B. et al. Pharmacol. Ther. 186, 73-87 (2018).
5. Cho, H., Blatchley, M. R., Duh, E. J. & Gerecht, S. Advanced Drug Delivery Reviews 50169-409X(18)30193-5 (2018). doi:10.1016/j.addr.2018.07.019
6. Gonzalez, A. C. de O., Costa, T. F., Andrade, Z. de A. & Medrado, A. R. A. P. An. Bras. Dermatol. 91, 614-620 (2016).
7. Oishi, Y. & Manabe, I. Int. Immunol. 30, 511-528 (2018).
8. Spees, J. L., Lee, R. H. & Gregory, C. A. Stem Cell Res. Ther. 7, 125 (2016).
9. Lai, R. C. et al. Stem Cell Res. 4, 214-222 (2010).
10. Philipp, D., Suhr, L., Wahlers, T., Choi, Y. H. & Paunel-Görgülü, A. Stem Cell Res. Ther. 9, 286 (2018).
11. Shafei, A. E.-S. et al. J. Gene Med. 19, e2995 (2017).

### 12. Theocharis, A. D., Skandalis, S. S., Gialeli, C. & Karamanos, N. K. Advanced Drug Delivery Reviews 97, 4-27 (2016).

13. Mulloy, B. & Rider, C. C. Biochem. Soc. Trans. 34, 409-413 (2006).
14. Wassenaar, J. W. et al. J. Am. Coll. Cardiol. 67, 1074-86 (2016).

### 15. Taylor, D. A., Sampaio, L. C., Ferdous, Z., Gobin, A. S. & Taite, L. J. Acta Biomater. 74, 74-89 (2018).

16. Mewhort, H. E. M. et al. J. Heart Lung Transplant. 35, 661-70 (2016).
17. Han, G. & Ceilley, R. Adv. Ther. 34, 599-610 (2017).

### 18. Aamodt, J. M. & Grainger, D. W. Biomaterials 86, 68-82 (2016).

19. Londono, R. et al. J. Biomed. Mater. Res. A 105, 2109-2118 (2017).
20. Daly, K. A. et al. Tissue Eng. Part A 18, 1293-303 (2012).
21. Badylak, S. F. & Gilbert, T. W. Semin. Immunol. 20, 109-116 (2008).

### 22. Badylak, S. F., Freytes, D. O. & Gilbert, T. W. Acta Biomater. 23 Suppl, S17-26 (2015).

23. Sun, Y. et al. FASEB J. 25, 1474-85 (2011).
24. Xiao, B. et al. Neural Regen. Res. 11, 1172-9 (2016).
25. Prewitz, M. C. et al. Biomaterials 73, 60-9 (2015).
26. Noh, Y. K. et al. Biomater. Res. 20, 6 (2016).
27. Lu, H. et al. Biomaterials 32, 9658-9666 (2011).
28. Sadr, N. et al. Biomaterials 33, 5085-93 (2012).
29. Deutsch, E. R. & Guldberg, R. E. J. Mater. Chem. 20, 8942 (2010).
30. Pham, Q. P. et al. J. Biomed. Mater. Res. A 88, 295-303 (2009).
31. Decaris, M. L., Binder, B. Y., Soicher, M. A., Bhat, A. & Leach, J. K. Tissue Eng. Part A 18, 2148-2157 (2012).
32. Fitzpatrick, L. E. & McDevitt, T. C. Biomater. Sci. 3, 12-24 (2015).
33. Chen, C., Loe, F., Blocki, A., Peng, Y. & Raghunath, M. Adv. Drug Deliv. Rev. 63, 277-90 (2011).
34. Ang, X. M. et al. Tissue Eng. Part A 20, 966-981 (2014).
35. Dewavrin, J. Y. et al. J. Phys. Chem. B 119, 4350-8 (2015).
36. Blocki, A. et al. Mol. Ther. 23, 510-522 (2015).
37. Blocki, A., Beyer, S., Jung, F. & Raghunath, M. Clin. Hemorheol. Microcirc. 69, 215-232 (2018).
38. Luo, R., Lu, Y., Liu, J., Cheng, J. & Chen, Y. Biomed. Pharmacother. 109, 2022-2034 (2019).
39. Chen, L. H. et al. Int. J. Biol. Macromol. 116, 572-584 (2018).
40. Longaker, M. T. et al. Ann. Surg. 210, 667-72 (1989).
41. Missinato, M. A., Tobita, K., Romano, N., Carroll, J. A. & Tsang, M. Cardiovasc. Res. 107, 487-498 (2015).
42. Anderegg, U., Simon, J. C. & Averbeck, M. Exp. Dermatol. 23, 295-303 (2014).

### 43. Crapo, P. M., Gilbert, T. W. & Badylak, S. F. Biomaterials 32, 3233-43 (2011).

44. Beyer, S., Koch, M., Lee, Y. H., Jung, F. & Blocki, A. Int. J. Mol. Sci 19, 2913 (2018).
45. Maxwell, S. Br. J. Clin. Pharmacol. 48, 284-98 (1999).
46. Hastings, C. L. et al. Adv. Drug Deliv. Rev. 84, 85-106 (2015).
47. Richardson, J. D. et al. Stem Cell Rev. Reports 9, 281-302 (2013).

### 48. Tzahor, E. & Poss, K. D. Science 356, 1035-1039 (2017).

### 49. Cahill, T. J., Choudhury, R. P. & Riley, P. R. Nature Reviews Drug Discovery 16, 699-717 (2017).

50. El Sayed Shafei, A. et al. J. Gene Med. 19, e2995 (2017).
51. Bronckaers, A. et al. Pharmacology and Therapeutics 143, 181-196 (2014).
52. Di Franco, S., Amarelli, C., Montalto, A., Loforte, A. & Musumeci, F. J. Thorac. Dis. 10, S2346-S2362 (2018).
53. Klopfleisch, R. & Jung, F. J. Biomed. Mater. Res. A 105, 927-940 (2017).

### 54. Kanji, S. & Das, H. Mediators Inflamm. 2017, 5217967 (2017).

55. Kim, H. J. & Park, J. S. Dev. Reprod. 21, 1-10 (2017).
56. Ho, J., Walsh, C., Yue, D., Dardik, A. & Cheema, U. Adv. wound care 6, 191-209 (2017).
57. Brittberg, M. et al. N. Engl. J. Med. 331, 889-895 (1994).
58. Torabi, A., Cleland, J. G., Rigby, A. S. & Sherwi, N. J. Geriatr. Cardiol. 11, 1-12 (2014).
59. Lee, V. W. Y. et al. Heart Asia 5, 1-6 (2013).

### 60. Leung, P. C. Surgeon 5, 219-31 (2007).

61. Raghav, A. et al. Ther. Adv. Endocrinol. Metab. 9, 29-31 (2018).
62. Chen, D. et al. Bone Res. 5, 16044 (2017).
63. Nelson, A. E. Osteoarthr. Cartil. 26, 319-325 (2018).
64. Available at: http://www.boneandjointburden.org. (Accessed: 2nd May 2019)
65. Lee, V. W. Y., Chan, W. K., Lam, N. L. C. & Lee, K. K. C. Dis. Manag. Heal. Outcomes 13, 281-285 (2005).

What is claimed is:

1. A method for producing an extracellular matrix material, comprising:
(1) culturing cells in the presence of an effective amount of a stimulant altering the cells' phenotype or bioactivity of the cell-derived extracellular matrix; and
(2) obtaining extracellular matrix material formed by the cells.

2. The method of claim 1, wherein the cells are stromal cells, stem cells, or progenitor cells.

3. The method of claim 1, wherein the cells are liver-derived cells, pancreas-derived cells, umbilical cord-derived cells, umbilical cord blood-derived cells, brain-derived cells, spleen-derived cells, bone marrow-derived cells, adipose-derived cells, cells derived from induced pluripotent stem cell (iPSC) technology, cells derived from embryonic stem cells, genetically engineered cells, pluripotent cells, multipotent cells, neural cells, astrocytes, hepatocytes, fibroblasts, mesenchymal cells, epithelial cells, endodermal cells, pericytes, cardiomyocytes, cardiomyocyte progenitor cells, hematopoietic cells, endothelial cells, endothelial progenitors, smooth muscle cells, keratinocytes, or mesenchymal stem/stromal cells.

4. The method of claim 1, wherein the stimulant is a glycosaminoglycan and/or a carbohydrate-based hydrophilic macromolecule.

5. The method of claim 4, wherein the glycosaminoglycan is heparan sulfate, chondroitin sulfate, dermatan sulfate, keratan sulfate, hyaluronic acid, proteoglycans carrying these glycosaminoglycans, derivates therefrom, combinations thereof.

6. The method of claim 5, wherein the glycosaminoglycan is hyaluronic acid.

7. The method of claim 6, wherein the hyaluronic acid has a molecular weight between about 2 kDa and about 10000 kDa, or between about 1500 kDa and about 1750 kDa.

8. The method of claim 6, wherein the hyaluronic acid is at a concentration of about 0.5 μg/ml to about 5000 μg/ml, about 5 μg/ml to about 1000 μg/ml, or about 500 μg/ml.

9. The method of claim 4, wherein the carbohydrate-based hydrophilic macromolecule is a polymer of glucose, sucrose, or a combination thereof.

10. The method of claim 9, wherein the polymer is Ficoll™70, Ficoll™400, polyvinyl pyrrolidone (PVP), dextran, dextran sulfate, polystyrene sulfonate, pullulan, chondroitan sulfate, heparin, heparan sulfate, dermatan sulfate, or a combination thereof.

11. The method of claim 4, wherein the carbohydrate-based hydrophilic macromolecules comprises Ficoll™70 and Ficoll™400.

12. The method of claim 10, wherein the Ficoll™70 is at a concentration of about 7.5 mg/ml to about 100 mg/ml, and the Ficoll™400 is at a concentration of about 2.5 mg/ml to about 100 mg/ml, or the Ficoll™70 is at a concentration of about 37.5 mg/ml and the Ficoll™400 is at a concentration of about 25 mg/ml.

13. The method of claim 10, wherein dextran sulfate is at a concentration of about 0.1 μg/ml to about 10 mg/ml or at a concentration of about 10 μg/ml.

14. The method of claim 1, wherein step (2) comprises decellularizing the extracellular matrix material.

15. The method of claim 14, wherein the decellularizing comprises lysis of cells present within the extracellular matrix material.

16. The method of claim 14, wherein the decellularizing comprises use of osmotic shock, freeze-thaw cycles, a lysing agent, or a combination thereof.

17. The method of claim 16, wherein the lysing agent is an ionic, non-ionic and non-denaturating, zwitterionic detergent, or chelating agent, nuclease, and a combination thereof.

18. The method of claim 17, wherein the lysing agent is deoxycholate, octylphenoxypolyethoxyethanol, 3-[(3-cholamidopropyl)dimethylammonio]-1-propanesulfonate (CHAPS), Ethylenediaminetetraacetic acid (EDTA), DNAse, or a combination thereof.

19. The method of claim 1, wherein step (2) comprises mechanical removal or solubilization of extracellular matrix material.

20. An extracellular matrix material produced by the method of claim 1, 13, or 14.

21. The extracellular matrix material of claim 20, wherein the extracellular matrix material has anti-inflammatory and/or pro-angiogenic properties.

22. A composition comprising (1) the extracellular matrix material of claims 20 and (2) a pharmaceutically acceptable excipient.

23. The composition of claim 22, wherein the extracellular matrix material has anti-inflammatory and/or pro-angiogenic properties.

24. The composition of claim 22, which is a solid, semi-solid, liquid, semi-liquid, emulsion, gel/hydrogel, microparticle, nanoparticle, capsule/microcapsule, film, patch, or bead/microbead.

25. A method for enhancing tissue healing and/or regeneration, comprising placing the extracellular matrix material of claim 20 or the composition of claim 22 at a site of tissue damage.

26. The method of claim 25, wherein the tissue damage is the result of an injury and/or a disease.

27. The method of claim 26, wherein the disease is biliary ischemia, bone-related ischemia, cerebral ischemia, colonic ischemia, coronary ischemia, foot-related ischemia, hepatic ischemia, mesenteric ischemia, myocardial ischemia, optical nerve ischemia, retinal ischemia and spinal ischemia, peripheral artery disease, myocardial infarction, chronic wounds, or osteoarthritis.

28. The method of claim 27, wherein the disease is myocardial infarction, chronic wounds, or osteoarthritis.
